(19) United States
(12) Patent Application Publication
(10) Pub. No.: US 2022/0033511 A1
(43) Pub. Date: Feb. 3, 2022

(54) METHODS OF MODULATING ANTIBODY-DEPENDENT CELL-MEDIATED CYTOTOXICITY

(71) Applicant: Amgen Inc., Thousand Oaks, CA (US)

(72) Inventors: Alla POLOZOVA, North Kingstown, RI (US); Qingchun ZHANG, Oak Park, CA (US); Scott KUHNS, Newbury Park, CA (US); Dong XIANG, Thousand Oaks, CA (US)

(21) Appl. No.: 17/275,140

(22) PCT Filed: Sep. 10, 2019
(86) PCT No.: PCT/US2019/050459

§ 371 Date: Mar. 10, 2021

Related U.S. Application Data
(60) Provisional application No. 62/729,971, filed on Sep. 11, 2018.

(51) Int. Cl. C07K 16/28; C07K 16/32; C07K 16/24

(52) U.S. Cl. CPC C07K 16/2887; C07K 16/32; C07K 16/241; C07K 2317/732; C07K 2317/72; C07K 2317/565; C07K 2317/14; C07K 2317/71; C07K 2317/41

(57) ABSTRACT

The present disclosure provides a method of controlling the Antibody Dependent Cellular Cytotoxicity (ADCC) activity of a glycosylated and afucosylated IgG1 antibody composition. In exemplary embodiments, the method includes (1) determining the ADCC activity of a glycosylated and afucosylated IgG1 antibody composition; and (2) increasing or decreasing the ADCC activity of the IgG1 antibody composition by increasing or decreasing the amount of terminal β-galactose in the afucosylated glycan species at the consensus glycosylation site. Related methods of matching ADCC activity of a reference glycosylated and afucosylated IgG1 antibody composition and methods of engineering a specific target ADCC activity of a glycosylated and afucosylated IgG1 antibody composition are further provided herein.

### FIELD OF THE INVENTION

The present invention relates generally to modulating Antibody-Dependent Cell-Mediated Cytotoxicity (ADCC) effector function of antibodies, e.g., IgG1 antibodies, including glycosylated and afucosylated IgG1 antibodies.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 28.6 KB ASCII (Text) file named “A-2246-WO-PCT_Final_Seqlisting_09092019.txt”; created on Sep. 9, 2019.

### BACKGROUND

Monoclonal antibody (mAb) based therapeutics have been effectively used to treat various diseases, such as cancers and chronic diseases. Many of these antibodies are of the immunoglobin G1s (IgG1s) subclass, which are often chosen because they have known effector function activities. IgGs have N-linked glycans at a conserved Asn residue in CH2 region of the mAb. Glycosylation at this site does not directly influence the target binding of a mAb, but can have significant impact on antibody effector functions, including antibody dependent cell-mediated cytotoxicity (ADCC), complement dependent cytotoxicity (CDC) and antibody dependent cellular phagocytosis (ADCP) (see Jefferis, R., Glycosylation as a strategy to improve antibody-based therapeutics. Nat Rev Drug Discov, 2009. 8(3): p. 226-34; Natsume, A., et. al, Improving effector functions of antibodies for cancer treatment: Enhancing ADCC and CDC. Drug Des Devel Ther, 2009. 3: p. 7-16), which can be critical for the mechanism of action (MOA) of some mAbs. Thus, there is a potential risk that the efficacy of a therapeutic antibody could fluctuate depending on the level and type of a particular glycan species present in a specific manufacturing lot.

Despite recent advances in bioreactor control and bioprocessing, it remains challenging to produce mAbs with well-defined glycan species using standard mAb production processes. Multiple factors can influence glycan profiles associated with recombinant antibodies. The high degree of heterogeneity and complexity inherent in Fc glycan structures associated with mAbs when they are produced with mammalian hosts is one of the main reasons. Flynn, G. C., et al., Naturally occurring glycan forms of human immunoglobulins G1 and G2. Mol Immunol, 2010. 47(11-12): p. 2074-82; Read, E. K., et al., Industry and regulatory experience of the glycosylation of monoclonal antibodies. Biotechnol Appl Biochem, 2011. 58(4): p. 213-9. In addition, batch to batch glycan profile variations of mAbs could arise from cellular changes including: the presence and concentration of processing enzymes, cell media components, kinetic parameters, availability of nucleotide sugar donors, etc. The intrinsic protein property could also affect glycan processing and result in different glycan structures. Dicker, M. and R. Strasser, Using glyco-engineering to produce therapeutic proteins. Expert Opin Biol Ther, 2015. 15(10): p. 1501-16. Therefore, from a therapeutic manufacturing viewpoint, there is much to be gained from an in-depth investigation of the impact of different glycoforms on immune cell mediated effector functions. Increasing knowledge of these relevant glycan species can be used to guide attribute-focused control strategies to ensure the control of critical attributes, and to allow appropriate flexibility in ranges for non-critical attributes.

As one of the key effector mechanisms underlying the clinical efficacy of some therapeutic antibodies, ADCC relies on the binding of cell surface antigen-antibody complexes to FcγIIIa receptors expressed on immune cells, which triggers the release of cytokines and cytotoxic granules that result in target cell death. ADCC activity in vitro is dependent on several parameters such as density of antigen on the surface of target cells, antigen-antibody affinity, and engagement of the complex to FcγR receptors, etc. For a target cell with desired antibody/antigen binding properties, ADCC activity will be highly dependent on the glycosylation profile of the Fc portion of a mAb owing to its influence on FcγIIIa receptor binding. Ferrara, C., et al., Unique carbohydrate-carbohydrate interactions are required for high affinity binding between FcgammaRIII and antibodies lacking core fucose. Proc Natl Acad Sci USA, 2011. 108(31): p. 12669-74; Okazaki, A., et al., Fucose depletion from human IgG1 oligosaccharide enhances binding enthalpy and association rate between IgG1 and FcgammaRIIIa. J Mol Biol, 2004. 336(5): p. 1239-49; Shields, R. L., et al., Lack of fucose on human IgG1 N-linked oligosaccharide improves binding to human Fcgamma RIII and antibody-dependent cellular toxicity. J Biol Chem, 2002. 277(30): p. 26733-40; Shinkawa, T., et al., The absence of fucose but not the presence of galactose or bisecting N-acetylglucosamine of human IgG1 complex-type oligosaccharides shows the critical role of enhancing antibody-dependent cellular cytotoxicity. J Biol Chem, 2003. 278(5): p. 3466-73. Therefore, owning to the potential impact to efficacy, glycosylation control has been identified as a key strategy in the manufacture of antibody based biotherapeutics. Jefferis, R., Glycosylation as a strategy to improve antibody-based therapeutics. Nat Rev Drug Discov, 2009. 8(3): p. 226-34. The effect of different types of Fc glycan structures on FcγR binding and ADCC activity has been investigated and several key relationships established. The absence of core fucose (also known as afucosylation) on complex glycans tends to enhance the binding affinity between mAbs and the FcγIIIa receptor and leads to increased ADCC activities. Ferrara, C., et al., Unique carbohydrate-carbohydrate interactions are required for high affinity binding between FcgammaRIII and antibodies lacking core fucose. Proc Natl Acad Sci USA, 2011. 108(31): p. 12669-74; Okazaki, A., et al., Fucose depletion from human IgG1 oligosaccharide enhances binding enthalpy and association rate between IgG1 and FcgammaRIIIa. J Mol Biol, 2004. 336(5): p. 1239-49; Shields, R. L., et al., Lack of fucose on human IgG1 N-linked oligosaccharide improves binding to human Fcgamma RIII and antibody-dependent cellular toxicity. J Biol Chem, 2002. 277(30): p. 26733-40; Shinkawa, T., et al., The absence of fucose but not the presence of galactose or bisecting N-acetylglucosamine of human IgG1 complex-type oligosaccharides shows the critical role of enhancing antibody-dependent cellular cytotoxicity. J Biol Chem, 2003. 278(5): p. 3466-73. High mannose glycans, which naturally lack core fucose, have also been shown to lead to higher ADCC activity (Kanda, Y., et al., Comparison of biological activity among nonfucosylated therapeutic IgG1 antibodies with three different N-linked Fc oligosaccharides: the high-mannose, hybrid, and complex types. Glycobiology, 2007. 17(1): p. 104-18; Pace, D., et al., Characterizing the effect of multiple Fc glycan attributes on the effector functions and FcgammaRIIIa receptor binding activity of an IgG1 antibody. Biotechnol Prog, 2016. 32(5): p. 1181-1192; Zhou, Q., et al., Development of a simple and rapid method for producing non-fucosylated oligomannose containing antibodies with increased effector function. Biotechnol Bioeng, 2008. 99(3): p. 652-65), whereas terminal sialyation has been found to decrease antibody binding to the FcγIIIa receptor and resulted in decreased ADCC activity (Kaneko, Y., F. et. al, Anti-inflammatory activity of immunoglobulin G resulting from rom Fc sialylation. Science, 2006. 313(5787): p. 670-3; Scallon, B. J., et al., Higher levels of sialylated Fc glycans in immunoglobulin G molecules can adversely impact functionality. Mol Immunol, 2007. 44(7): p. 1524-34).

The understanding of the impact of terminal galactosylation of Fc glycans on ADCC remains an active area of investigation. Some studies suggest that terminal galactose has no effect on the binding of mAbs to FcγIIIa and ADCC activity (Shinkawa, T., et al., The absence of fucose but not the presence of galactose or bisecting N-acetylglucosamine of human IgG1 complex-type oligosaccharides shows the critical role of enhancing antibody-dependent cellular cytotoxicity. J Biol Chem, 2003. 278(5): p. 3466-73; Boyd, P. N., et al, The effect of the removal of sialic acid, galactose and total carbohydrate on the functional activity of Campath-1H. Mol Immunol, 1995. 32(17-18): p. 1311-8; Hodoniczky, J., et. al, Control of recombinant monoclonal antibody effector functions by Fc N-glycan remodeling in vitro. Biotechnol Prog, 2005. 21(6): p. 1644-52; Raju, T. S., Terminal sugars of Fc glycans influence antibody effector functions of IgGs. Curr Opin Immunol, 2008. 20(4): p. 471-8), while other studies indicate that Fc galactosylation can have a positive impact on such activity (Kumpel, B. M., et al., The biological activity of human monoclonal IgG anti-D is reduced by beta-galactosidase treatment. Hum Antibodies Hybridomas, 1995. 6(3): p. 82-8; Thomann, M., et al., Fc-galactosylation modulates antibody-dependent cellular cytotoxicity of therapeutic antibodies. Mol Immunol, 2016. 73: p. 69-75; Thomann, M., et al., In vitro glycoengineering of IgG1 and its effect on Fc receptor binding and ADCC activity. PLoS One, 2015. 10(8): p. e0134949; Houde, D., et al., Post-translational modifications differentially affect IgG1 conformation and receptor binding. Mol Cell Proteomics, 2010. 9(8): p. 1716-28. An in-depth understanding of the relationship between galactosylation and ADCC activities of mAbs offers opportunity to design and produce therapeutic mAbs with desired therapeutic properties and to optimize control strategies.

### SUMMARY

Herein we demonstrate that terminal β-galactose significantly influences ADCC activity of glycosylated and afucosylated IgG1 antibodies. Accordingly, the present disclosure provides methods of modulating (i.e. increasing or decreasing) ADCC activity of a glycosylated and afucosylated IgG1 antibody composition (including methods of increasing or decreasing ADCC activity of a composition comprising a glycosylated and afucosylated anti-HER2 antibody, anti-TNFα, or anti-CD20 antibody, including trastuzumab, infliximab or rituximab) by modulating (i.e., increasing or decreasing) terminal β-galactose (including, e.g., enriching, increasing, removing and/or remodeling galactosylated glycans). In exemplary embodiments, the method of modulating ADCC activity of a glycosylated and afucosylated IgG1 antibody composition (such as a composition comprising an anti-HER2 antibody, an anti-TNFα, or an anti-CD20 antibody, including trastuzumab, infliximab or rituximab) comprises modulating the amount of terminal galactose on one or more IgG1 antibodies within the composition, e.g., increasing the amount of terminal galactose on one or more IgG1 antibodies within the composition to increase ADCC activity or decreasing the amount of terminal galactose on one or more IgG1 antibodies within the composition to decrease ADCC activity.

In exemplary embodiments, the method of modulating ADCC activity comprises modulating the amount or percentage of afucosylated, galactosylated IgG1 antibodies of an antibody composition (such as an anti-HER2 antibody, an anti-TNFα, or an anti-CD20 antibody, including trastuzumab, infliximab or rituximab). In exemplary aspects, the methods provided herein increase ADCC activity by increasing the amount or percentage of afucosylated, galactosylated IgG1 antibodies of an antibody composition (such as an anti-HER2 antibody, an anti-TNFα, or an anti-CD20 antibody, including trastuzumab, infliximab or rituximab). In alternative exemplary aspects, the methods provided herein decrease ADCC activity by decreasing the amount or percentage of afucosylated, galactosylated IgG1 antibodies of an antibody composition (such as an anti-HER2 antibody, an anti-TNFα, or an anti-CD20 antibody, including trastuzumab, infliximab or rituximab).

The present disclosure provides methods of controlling, modulating or maintaining the ADCC activity of an antibody composition comprising glycosylated and afucosylated IgG1 antibodies (such as anti-HER2 antibodies, anti-TNFα, or anti-CD20 antibodies, including trastuzumab, infliximab or rituximab). In exemplary embodiments, the method comprises: (1) determining the ADCC activity of a composition comprising glycosylated and afucosylated IgG1 antibodies (such as anti-HER2 antibodies, anti-TNFα, or anti-CD20 antibodies, including trastuzumab, infliximab or rituximab); and (2) increasing or decreasing the ADCC activity of the IgG1 antibody composition by increasing or decreasing the amount of terminal β-galactose in the glycan species at the consensus glycosylation site of one or more antibodies within the composition.

The present disclosure also provides a method of matching the ADCC activity of a reference composition comprising glycosylated and afucosylated IgG1 antibodies (such as anti-HER2 antibodies, anti-TNFα, or anti-CD20 antibodies, including trastuzumab, infliximab or rituximab). In exemplary embodiments, the method comprises: (1) determining the ADCC activity of a reference glycosylated and afucosylated IgG1 antibody composition; (2) determining the ADCC activity of a second antibody composition comprising an IgG1 antibody having the same antibody sequence as the reference IgG1 antibody; and (3) changing the ADCC activity of the second antibody composition by increasing or decreasing the amount of terminal β-galactose in the glycan species at the consensus glycosylation site of one or more antibodies within the second antibody composition, wherein the ADCC activity of the second antibody composition after increasing or decreasing the amount of terminal β-galactose is the same as the reference IgG1 antibody composition or within about 10%, about 15%, about 20%, about 25%, about 30%, about 35%, about 40%, about 45% or about 50% of the reference IgG1 antibody composition or within about 1% to about 50% of the reference IgG1 antibody composition. In some embodiments, step 1 (“determining the ADCC activity of a reference glycosylated and afucosylated IgG1 antibody composition”) occurs before, after or at the same time as step 2 (“determining the ADCC activity of a second antibody composition comprising an IgG1 antibody having the same antibody sequence as the reference IgG1 antibody”) and/or step 3 (“changing the ADCC activity of the second antibody composition . . . ”). Also provided by the present disclosure is a method for engineering a specific target ADCC activity of a composition comprising glycosylated and afucosylated IgG1 antibodies (such as anti-HER2 antibodies, anti-TNFα, or anti-CD20 antibodies, including trastuzumab, infliximab or rituximab). In exemplary embodiments, the method comprises: (1) determining the ADCC activity of a composition comprising glycosylated and afucosylated IgG1 antibodies; (2) determining a target ADCC activity; and (3) increasing or decreasing the ADCC activity of the IgG1 antibody composition by increasing or decreasing the amount of terminal β-galactose in the glycan species at the consensus glycosylation site of one or more antibodies within the composition, wherein the ADCC activity of the antibody composition after increasing or decreasing the amount of terminal β-galactose is the same as the target ADCC activity or within about 10%, about 15%, about 20%, about 25%, about 30%, about 35%, about 40%, about 45% or about 50% of the target ADCC activity or within about 1% to about 50% of the target ADCC activity. In some embodiments, step 2 (“determining a target ADCC activity”) occurs before, after or at the same time as step 1 (“determining the ADCC activity of a composition comprising glycosylated and afucosylated IgG1 antibody”) and/or step 3 (“increasing or decreasing the ADCC activity of the IgG1 antibody . . . ”). In some other embodiments, step 1 (“determining the ADCC activity of a composition comprising glycosylated and afucosylated IgG1 antibodies”) occurs before, after or at the same time as step 2 (“determining a target ADCC activity”) and/or step 3 (“increasing or decreasing the ADCC activity of the IgG1 antibody composition . . . ”).

### BRIEF DESCRIPTION OF THE DRAWINGS

FIG. 1 is an illustration of the three major types of N-glycans commonly found on mammalian proteins (oligomannose, complex and hybrid) and commonly used symbols for such glycans. In CHO produced monoclonal IgG antibodies, level of terminal sialic acid is usually low and oligosaccharides with terminal galactose, GlcNac or mannose are more prevalent.

FIG. 2 is a schematic representation of key glycan group classifications. The glycan structures shown in each group are not fully comprehensive, i.e., only representative structures, typical of CHO-expressed IgG.

FIG. 3A is an illustration of a crystal structure of IgG1 Fc region complexed with FcγRIIIa receptor binding site (from Mizushima et al. Genes to Cells (2011) 16, 1071-1080).

FIG. 3B is an illustration of a structural hypothesis of more optimal and higher affinity binding for afucosylated galactosylated glycan species.

FIGS. 4A and 4B are graphs showing a glycan-ADCC model based on a combination of contributions from afucosylated galactosylated and afucosylated agalactosylated species. FIG. 4A is an assessment of model fit and FIG. 4B is a graph depicting contributions (leverage) of individual components. FIG. 4C depicts an example of ADCC target range supported by a combination of contributions from afucosylated galactosyated and afucosylated agalactosylated glycan groups.

FIG. 5 is a diagram of the salvage pathway and the de novo pathway of fucose metabolism. In the salvage pathway, free L-fucose is converted to GDP-fucose, while in the de novo pathway, GDP-fucose is synthesized via three reactions catalyzed by GMD and FX. GDP-fucose is then transported from the cytosol to the Golgi lumen by GDP-Fuc Transferase and transferred to acceptor oligosaccharides and proteins. The other reaction product, GDP, is converted by a luminal nucleotide diphosphatase to guanosine 5-monophosphate (GMP) and inorganic phosphate (Pi). The former is exported to the cytosol (via an antiport system that is coupled with the transport of GDP-fucose), whereas the latter is postulated to leave the Golgi lumen via the Golgi anion channel, G0LAC. See, e.g., Nordeen et al. 2000; Hirschberg et al. 2001.

FIG. 6 demonstrates the effect of total galactosylation on ADCC activities for (A) an anti-HER2 IgG1 antibody (trastuzumab) (“mAb1”), (B) an anti-CD20 IgG1 antibody (rituximab) (“mAb2”), and (C) an anti-TNFα IgG1 antibody (infliximab) (“mAb3”). In vitro enzymatic remodeling of drug substances of all three antibodies was performed to generate samples with a wide range of different levels of galactosylated mAbs, while other glycan attributes such as afucosylation (Afuc %) and high mannose (HM %) were held constant for each individual mAb. FIG. 6A is a graph of the relative ADCC activity (%) plotted as a function of % Gal of an anti-HER2 IgG1 antibody (trastuzumab) composition and the table below the graph lists the glycan profile of the trastuzumab antibody composition. FIG. 6B is a graph of the relative ADCC activity (%) plotted as a function of % Gal of an anti-CD20 IgG1 antibody (rituximab) composition and the table below the graph lists the glycan profile of the rituximab antibody composition. FIG. 6C is a graph of the relative ADCC activity (%) plotted as a function of % Gal of an anti-TNFα IgG1 antibody (infliximab) composition and the table below the graph lists the glycan profile of the infliximab antibody composition.

FIG. 7 demonstrates antigen binding activity for (A) an anti-HER2 IgG1 antibody (trastuzumab) (“mAb1”) and (B) an anti-CD20 IgG1 antibody (rituximab) (“mAb2”) with different levels of terminal galactose. Relative activities shown here were normalized to the activity of the samples with lowest galactose levels for each mAb. FIG. 7A is a graph of the relative target binding (%) plotted for a trastuzumab antibody composition comprising 1% Gal, 52% Gal, or 91% Gal. FIG. 7B is a graph of the relative target binding (%) plotted for a rituximab antibody composition comprising 0% Gal, 53% Gal, or 89% Gal.

FIG. 8 is an illustration of an in vitro glycan enrichment workflow to generate antibodies with G0F, G1 and G0 enriched species to study the impact of galactosylation on mAbs with afucosylated glycan structures. FcγIIIa receptor affinity chromatography was used to separate fucosylated species from afucosylated and high mannose species. Galactose in the fucosylated fraction was removed using galactosidase to generate mAbs with G0F as the dominant glycoform. Afucosylated species were further enriched by first removing high mannose with endo-H treatment in the eluted fraction from the FcγIIIa receptor column, followed by treatment with galactosidase to generate afucosylated G0 and G1 samples. Intact mass analysis of mAbs was conducted to closely monitor each step and the enriched materials were further characterized.

FIG. 9 demonstrates the effect of terminal Gal associated with afucosylated glycans on ADCC activity for an anti-HER2 IgG1 antibody (trastuzumab). FIG. 9A is a table listing the percentage of G0 and G1 species in G0F enriched, G0 enriched and G1 enriched samples and an illustration below the table depicting a cartoon of the G0F, G0, and G2 glycans. FIG. 9B is a graph of the relative ADCC activities (%) for initial drug substance (“DS”), G0F, G0 series (G0-1, G0-2 & G0-3), and G1 series (G0-1, G0-2 & G0-3) samples. The grey bars represent the ADCC activities for G0 series of samples while the patterned bars grey bars represent the ADCC activities for G1 series samples. The starting material DS and the enriched G0F are two controls (black bars). FIG. 9C is a pair of graphs of the relative ADCC activities (%) as a function of G0(%) (top) or G1(%) (bottom) for trastuzumab. The G0 impact on ADCC (FIG. 9C top panel) was readily obtained from G0 series samples as G0 is the main afucosylated species. The impact of G1 was calculated by removing the G0 contribution from G1 series based on the G0 impact coefficiency from FIG. 9C, top panel.

FIG. 10 demonstrates the experimental measurement of the total afucosylation impact on ADCC activity of an anti-HER2 IgG1 antibody (trastuzumab). The trastuzumab DS lot containing both afucosylated G1 and G0 species, was treated with Endo-H followed by affinity chromatography to enrich afucosylated mAb. The afucose enriched trastuzumab was blended with the G0F enriched trastuzumab at different ratios followed by ADCC activity measurement to assess the overall impact of both species on ADCC activities. FIG. 10 is a graph of the relative ADCC activity (%) as a function of afucosylated glycans (%).

FIG. 11 demonstrates the effect of terminal Gal associated with afucosylated glycans on ADCC activity for an anti-CD20 IgG1 antibody (rituximab). FIG. 11A is a graph of the relative ADCC activities for initial drug substance (“DS”), G0F, G0 series (G0-1, G0-2 & G0-3), and G1 series (G0-1, G0-2 & G0-3) samples. The grey bars represent the ADCC activities for G0 series of samples while the patterned bars grey bars represent the ADCC activities for G1 series samples. The starting material DS and the enriched G0F are two controls (black bars). Below the graph is a table listing the amounts of the glycan species for each sample or sample series. FIG. 11B is a pair of graphs showing the correlation of G0% (top) and G1% (bottom) with ADCC activity for rituximab. The G0 impact on ADCC (FIG. 11 top panel) was readily obtained from G0 series samples as G0 is the main afucosylated species. The impact of G1 was calculated by removing the G0 contribution from G1 series based on the G0 impact coefficiency from FIG. 11B, top panel.

FIG. 12 demonstrates the effect of terminal Gal associated with fucosylated glycans on ADCC activity for (A) an anti-HER2 IgG1 antibody (trastuzumab) (“mAb1”) and (B) an anti-CD20 IgG1 antibody (rituximab) (“mAb2”). Assessment of terminal galactose impact on ADCC activities for fucosylated trastuzumab and rituximab was performed by generating G0F enriched samples for each mAb as described in FIG. 8 (left) followed by enzymatic remodeling with β (1, 4) galactosyltransferase. FIG. 12A is a graph of the relative ADCC activity (%) as a function of Gal (%) in the sample containing trastuzumab and FIG. 12B is a graph of the relative ADCC activity (%) as a function of Gal (%) in the sample containing rituximab.

### DETAILED DESCRIPTION

In order that the present disclosure can be more readily understood, certain terms are first defined. Additional definitions are set forth throughout the detailed description.

As used herein, the terms “a,” “an,” and “the” and similar referents are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,” and permit the presence of one or more features or components) unless otherwise noted. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used in connection with a numerical value or range throughout the specification and the claims denotes an interval of accuracy, familiar and acceptable to a person skilled in the art. In general, such interval of accuracy is ±10%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure. Generally, nomenclatures used in connection with, and techniques of, cell and tissue culture, molecular biology, immunology, protein glycosylation, antibody production and antibody purification, described herein are those well-known and commonly used in the art. Standard techniques may be used for recombinant DNA, oligonucleotide synthesis, tissue culture and transformation, protein purification, antibody generation, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manual, 3rd ed., Cold Spring Harbor Laboratory Press, cold Spring Harbor, N.Y., which is incorporated herein by reference for any purpose.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole.

### Post-Translational Glycosylation

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or O-linked), microheterogeneity of protein glycoforms exists due to the large range of glycan structures associated with each site (0 or N).

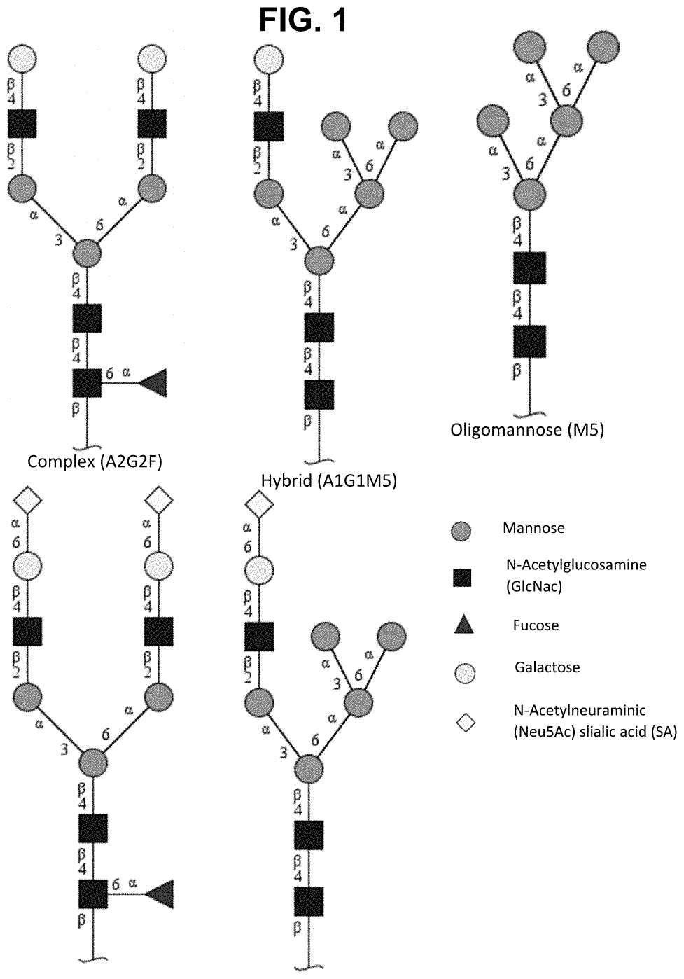

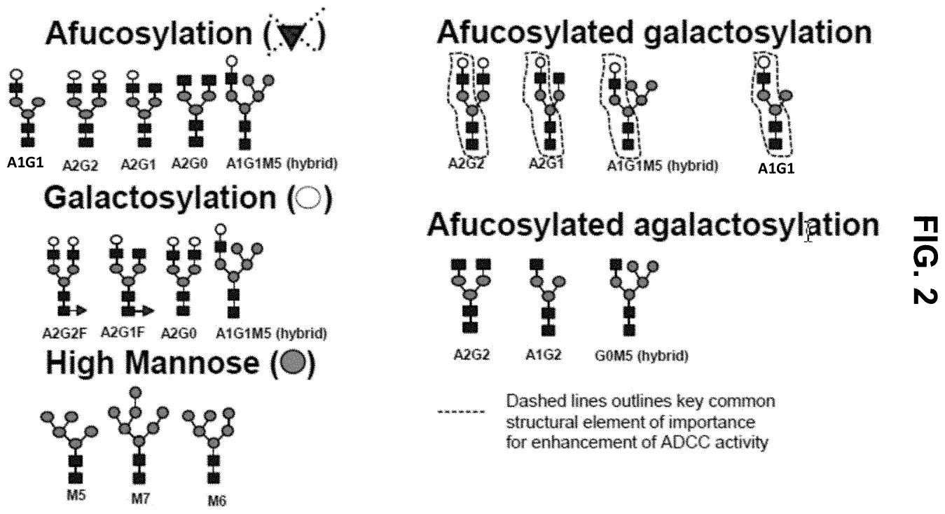

All N-glycans have a common core sugar sequence: Manα1-6(Manα1-3)Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and a large number (e.g., 4, 5, 6, 7, 8 or 9) of mannose (Man) residues (B) a complex type, which comprises more than two GlcNAc moieties and any number of other sugar types or (C) a hybrid type, which comprises a Man residue(s) on one side of the branch and GlcNAc at the base of a complex branch. FIG. 1A (taken from Stanley et al., Chapter 8: N-Glycans, Essentials of Glycobiology, 2nd ed., Cold Spring Harbor Laboratory Press; 2009) shows the three types of N-glycans.

N-linked glycans typically comprise one or more monosaccharides of galactose (Gal), N-acetylgalactosamine (GalNAc), N-acetylglucoasamine (GlcNAc), mannose (Man), NOAcetylneuraminic acid (Neu5Ac), fucose (Fuc). The commonly used symbols for such saccharides are shown in FIG. 1A.

N-linked glycosylation begins in the endoplasmic reticulum (ER), where a complex set of reactions result in the attachment of a core glycan structure comprised of two GlcNAc and three Man units. Additional Man units can be added to the core glycan structure upon further processing resulting in high mannose (HM) structures. The glycan complex formed in the ER is modified by action of enzymes in the Golgi apparatus. If the oligosaccharide is relatively inaccessible to the enzymes or enzymes are absent or unactive, the oligosaccharide will remain in the original HM form. If active enzymes can access the oligosaccharide, then the non-core Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84).

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

Controlling the glycan structure is important in recombinant production of therapeutic monoclonal antibodies, as the glycan structure attached to the Fc domain influences the interaction with the FcγRs that mediate ADCC and ADCP and with C1q binding, the initial binding event leading to CDC.

ADCC has been identified as one of the potentially critical effector functions underlying the clinical efficacy of some therapeutic IgG1 antibodies. It has been well established that higher levels of afucosylated N-linked glycan structures on the Fc region enhance the IgG binding affinity to the FcγIIIa receptor and lead to increased ADCC activity. However, whether terminal galactosylation of an IgG1, including afucosylated IgG1s, impacts ADCC activity is less clear.

Here, a strategy was used for analysis of relationships between the glycan composition and ADCC function to identify the active species in the IgG1 compositions with varying ranges of ADCC. The results presented herein indicate that the degree of influence of terminal β-galactose on in vitro ADCC activity depends on the absence of the core fucose, which is typically linked to the first N-acetyl glucosamine residue of an N-linked glycosylation core structure. Additionally, glycan enrichment and blending studies were performed to confirm the impact of terminal β-galactose on ADCC activity for therapeutic IgG1 compositions and the results were consistent with the glycan composition—ADCC modeling observations. Specifically, terminal β-galactose on afucosylated mAbs enhanced ADCC activity but did not impact activities on fucosylated glycan structures. Knowledge gained here not only can be used to guide product and process development activities for biotherapeutic antibodies that require effector function for efficacy, but also highlights the level of complexity in modulating the immune response through N-linked glycosylation of antibodies.

Accordingly, the present disclosure describes the impact of terminal β-galactose on ADCC activity of glycosylated and afucosylated IgG1 antibodies, including, e.g., trastuzumab, rituximab or infliximab, and thus provides methods of modulating (i.e. increasing or decreasing) ADCC activity of glycosylated and afucosylated IgG1 antibody compositions (including methods of increasing or decreasing ADCC activity of an anti-HER2 antibody composition, an anti-TNFα, antibody composition, or an anti-CD20 antibody composition, including those containing trastuzumab, infliximab or rituximab) by modulating (i.e., increasing or decreasing) terminal β-galactose (including, e.g., enriching, increasing, removing and/or remodeling galactosylated glycans) within the composition. The present disclosure also provides methods of modulating ADCC activity induced or stimulated by an IgG1 antibody composition (such as an anti-HER2 antibody, an anti-TNFα antibody, or an anti-CD20 antibody, including trastuzumab, infliximab or rituximab), comprising modulating (i.e., increasing or decreasing) the amount of galactosylated glycoforms, afucosylated glycoforms, or a combination thereof (e.g., galactosylated afucosylated glycoforms) within the antibody composition. In exemplary aspects, increasing the amount of galactosylated glycoforms, afucosylated glycoforms, or a combination thereof (e.g., galactosylated afucosylated glycoforms) within the IgG1 antibody composition (such as an anti-HER2 antibody, an anti-TNFα antibody, or an anti-CD20 antibody, including trastuzumab, infliximab or rituximab) increases the ADCC activity of the antibody composition, while decreasing the amount of galactosylated glycoforms, afucosylated glycoforms, or a combination thereof (e.g., galactosylated afucosylated glycoforms) within the IgG1 antibody composition (such as an anti-HER2 antibody, an anti-TNFα antibody, or an anti-CD20 antibody, including trastuzumab, infliximab or rituximab) decreases the ADCC activity of the antibody composition.

In exemplary embodiments, the method of modulating ADCC activity of an IgG1 antibody (such as an anti-HER2 antibody, an anti-TNFα, or an anti-CD20 antibody, including trastuzumab, infliximab or rituximab) comprises modulating the presence or absence of terminal β-galactose on an IgG1 antibody, e.g., adding terminal β-galactose on the IgG1 antibody to increase ADCC activity or removing terminal β-galactose on the IgG1 antibody to decrease ADCC activity. In exemplary aspects, the IgG1 antibody is afucosylated. Accordingly, in exemplary aspects, the method of modulating ADCC activity of an IgG1 antibody (such as an anti-HER2 antibody, an anti-TNFα, or an anti-CD20 antibody, including trastuzumab, infliximab or rituximab) comprises adding terminal β-galactose to an afucosylated IgG1 antibody, e.g., an afucosylated IgG1 antibody (such as an anti-HER2 antibody, an anti-TNFα, or an anti-CD20 antibody, including trastuzumab, infliximab or rituximab) to increase its ADCC activity or removing terminal β-galactose from an afucosylated IgG1 antibody (such as an anti-HER2 antibody, an anti-TNFα, or an anti-CD20 antibody, including trastuzumab, infliximab or rituximab) to decrease its ADCC activity.

In exemplary embodiments, the method of modulating ADCC activity of a composition comprising an IgG1 antibody (such as an anti-HER2 antibody, an anti-TNFα, or an anti-CD20 antibody, including trastuzumab, infliximab or rituximab) comprises modulating the amount of galactosylated glycoforms of an afucosylated antibody composition, e.g., increasing the amount of galactosylated glycoforms on afucosylated antibodies within the composition to increase ADCC activity of the antibody composition, or decreasing the amount of galactosylated glycoforms on afucosylated antibodies within the composition to decrease ADCC activity of the antibody composition.

In exemplary embodiments, the method of modulating ADCC activity comprises modulating the amount or percentage of afucosylated, galactosylated IgG1 antibodies of an antibody composition (such as an anti-HER2 antibody, an anti-TNFα, or an anti-CD20 antibody, including trastuzumab, infliximab or rituximab). In exemplary aspects, the method increases ADCC activity by increasing the amount or percentage of afucosylated, galactosylated IgG1 antibodies (such as an anti-HER2 antibody, an anti-TNFα, or an anti-CD20 antibody, including trastuzumab, infliximab or rituximab). In alternative exemplary aspects, the method decreases ADCC activity by decreasing the amount or percentage of afucosylated, galactosylated IgG1 antibodies (such as an anti-HER2 antibody, an anti-TNFα, or an anti-CD20 antibody, including trastuzumab, infliximab or rituximab).

The present disclosure provides methods of controlling, modulating or maintaining the ADCC activity of a glycosylated and afucosylated IgG1 antibody composition. In exemplary embodiments, the method comprises: (1) determining the ADCC activity of a glycosylated and afucosylated IgG1 antibody composition; and (2) increasing or decreasing the ADCC activity of the IgG1 antibody composition by increasing or decreasing the amount or percentage of terminal β-galactose in the glycan species at the consensus glycosylation site of the afucosylated IgG1 antibodies within the composition.

The present disclosure also provides a method of matching the ADCC activity of a reference glycosylated and afucosylated IgG1 antibody composition. In exemplary embodiments, the method comprises: (1) determining the ADCC activity of a reference glycosylated and afucosylated IgG1 antibody composition; (2) determining the ADCC activity of a second composition comprising an antibody having the same antibody sequence as the reference IgG1 antibody; and (3) changing the ADCC activity of the second composition by increasing or decreasing the amount or percentage of terminal β-galactose in the glycan species at the consensus glycosylation site of the afucosylated IgG1 antibodies within the composition, wherein the ADCC activity of the second composition after increasing or decreasing the amount of terminal β-galactose is the same as the reference IgG1 antibody composition or within about 10%, about 15%, about 20%, about 25%, about 30%, about 35%, about 40%, about 45% or about 50% of the reference IgG1 antibody composition or within about 1% to about 50% of the reference IgG1 antibody composition. In some embodiments, step 1 (“determining the ADCC activity of a reference glycosylated and afucosylated IgG1 antibody composition”) occurs before, after or at the same time as step 2 (“determining the ADCC activity of a second composition comprising an antibody having the same antibody sequence as the reference IgG1 antibody”) and/or step 3 (“changing the ADCC activity of the second composition . . . ”).

Also provided by the present disclosure is a method for engineering a specific target ADCC activity of a glycosylated and afucosylated IgG1 antibody composition. In exemplary embodiments, the method comprises: (1) determining the ADCC activity of a glycosylated and afucosylated IgG1 antibody composition; (2) determining a target ADCC activity; and (3) increasing or decreasing the ADCC activity of the IgG1 antibody composition by increasing or decreasing the amount or percentage of terminal β-galactose in the glycan species at the consensus glycosylation site of the afucosylated IgG1 antibodies within the composition, wherein the ADCC activity of the antibody composition after increasing or decreasing the amount of terminal β-galactose is the same as the target ADCC activity or within about 10%, about 15%, about 20%, about 25%, about 30%, about 35%, about 40%, about 45% or about 50% of the target ADCC activity or within about 1% to about 50% of the target ADCC activity. In some embodiments, step 2 (“determining a target ADCC activity”) occurs before, after or at the same time as step 1 (“determining the ADCC activity of a glycosylated and afucosylated IgG1 antibody composition”) and/or step 3 (“increasing or decreasing the ADCC activity of the IgG1 antibody composition . . . ”). In some other embodiments, step 1 (“determining the ADCC activity of a glycosylated and afucosylated IgG1 antibody composition”) occurs before, after or at the same time as step 2 (“determining a target ADCC activity”) and/or step 3 (“increasing or decreasing the ADCC activity of the IgG1 antibody composition . . . ”).

The term “antibody-dependent cell-mediated cytotoxicity” or “ADCC” or “antibody-dependent cellular cytotoxicity” refers to the mechanism by which an effector cell of the immune system, principally natural killer cells (NK cells), actively lyses a target cell, whose membrane-surface antigens have been bound by specific antibodies. ADCC is a part of the adaptive immune response and occurs when antigen-specific antibodies bind to (1) the membrane-surface antigens on a target cell through its antigen-binding regions and (2) to Fc receptors, principally FcγRIIIa (CD 16), on the surface of the effector cells through its Fc region. Binding of the Fc region of the antibody to the Fc receptor causes the effector cells to release cytotoxic factors that lead to death of the target cell (e.g., through cell lysis or cellular degranulation).

Fc receptors are receptors on the surfaces of B lymphocytes, follicular dendritic cells, NK cells, macrophages, neutrophils, eosinophils, basophils, platelets and mast cells that bind to the Fc region of an antibody. Fc receptors are grouped into different classes based on the type of antibody that they bind. For example, an Fc-gamma receptor is a receptor for the Fc region of an IgG antibody, an Fc-alpha receptor is a receptor for the Fc region of an IgA antibody, and an Fc-epsilon receptor is a receptor for the Fc region of an IgE antibody.

The term “FcγR” or “Fc-gamma receptor” is a protein belonging to the immunoglobulin superfamily involved in inducing phagocytosis of opsonized cells or microbes. See, e.g., Fridman W H. Fc receptors and immunoglobulin binding factors. FASEB Journal. 5 (12): 2684-90 (1991). Members of the Fc-gamma receptor family include: FcγRI (CD64), FcγRIIA (CD32), FcγRIIB (CD32), FcγRIIIA (CD16a), and FcγRIIIB (CD16b). The sequences of FcγRI, FcγRIIA, FcγRIIB, FcγRIIIA, and FcγRIIIB can be found in many sequence databases, for example, at the Uniprot database (www.uniprot.org) under accession numbers P12314 (FCGR1_HUMAN), P12318 (FCG2A_HUMAN), P31994 (FCG2B_HUMAN), P08637 (FCG3A_HUMAN), and P08637 (FCG3A_HUMAN), respectively.

The term “ADCC activity” refers to the extent to which ADCC is activated or stimulated. The phrase “ADCC activity of an antibody” refers to the ability of an antibody to induce ADCC.

Methods of measuring or determining the ADCC activity of an antibody or antibody composition, including commercially available assays and kits, are well-known in the art, as described, Yamashita et al., Scientific Reports 6: article number 19772 (2016); Kantakamalakul et al., “A novel EGFP-CEM-NKr flow cytometric method for measuring antibody dependent cell mediated-cytotoxicity (ADCC) activity in HIV-1 infected individuals”, J Immunol Methods 315(Issues 1-2): 1-10; (2006); Gomez-Roman et al., “A simplified method for the rapid fluorometric assessment of antibody-dependent cell-mediated cytotoxicity”, J Immunol Methods 308 (Issues 1-2): 53-67 (2006); Schnueriger et al., Development of a quantitative, cell-line based assay to measure ADCC activity mediated by therapeutic antibodies, Molec Immunology 38 (Issues 12-13): 1512-1517 (2011); and Mata et al., “Effects of cryopreservation on effector cells for antibody dependent cell-mediated cytotoxicity (ADCC) and natural killer (NK) cell activity in ⁵¹Cr-release and CD107a assays”, J Immunol Methods 406: 1-9 (2014); all herein incorporated by reference for all purposes. The term “ADCC Assay” or “FcγR reporter gene assay” refers to an assay, kit or method useful to determine the ADCC activity of an antibody or antibody composition.

Exemplary methods of measuring or determining the ADCC activity of an antibody composition in the methods described herein include the ADCC assay described in the Examples or the ADCC Reporter Assay commercially available from Promega (Catalog No. G7010 and G7018). In some embodiments, ADCC activity is measured or determined using a calcein release assay containing one or more of the following: a FcγRIIIa (158V)-expressing NK92(M1) cells as effector cells and HCC2218 cells or WIL2-S cells as target cells labeled with calcein-AM.

### Modulating ADCC Activity

The term “modulate” or “modulating” means to change by increasing or decreasing. Thus, the term “modulating” as used in a phrase such as “modulating ADCC activity” herein is intended to include increasing ADCC activity or decreasing ADCC activity. Also, the term “modulating” as used in a phrase such as “modulating the amount of galactosylated, afucosylated glycans, fucosylated glycans, galactosylated glycans, afucosylated glycans, or a combination thereof” is intended to include increasing the amount of said glycans or decreasing the amount of said gly cans.

Accordingly, in exemplary embodiments, the presently disclosed method represents a method of increasing ADCC activity of an antibody or a composition comprising the same. In exemplary aspects, the methods of the present disclosure increase the ADCC activity of the antibody, or composition comprising the same, to any degree or level relative to a control or a reference antibody. In exemplary instances, the increase in ADCC activity provided by the methods of the disclosure is at least or about a 1% to about a 100% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, at least or about a 100% increase) relative to a control or a reference antibody. In exemplary embodiments, the increase provided by the methods of the disclosure is over 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody. In exemplary embodiments, the level of ADCC activity of the antibody or composition comprising the same increases by an amount falling within the range of about 5% to about 400%, relative to a control or a reference antibody. In exemplary embodiments, the level of ADCC activity of the antibody or composition comprising the same increases by at least or about 1.5-fold, by at least or about 2-fold, by at least or about 3-fold, by at least or about 4-fold or by at least or about 5-fold, relative to a control or a reference antibody. In exemplary embodiments, the level of ADCC activity of the antibody or composition comprising the same increases by at about 6-fold, about 7-fold, about 8-fold, about 9-fold, or about 10-fold, relative to a control or a reference antibody. In exemplary embodiments, the level of ADCC activity of the antibody or composition comprising the same increases by an amount falling within the range of about 0.5-fold to about 8-fold, relative to a control or a reference antibody.

In alternative embodiments, the presently disclosed method represents a method of decreasing ADCC activity of an antibody or a composition comprising the same. In some aspects, the methods of the disclosure decrease the level of ADCC activity of the antibody, or composition comprising the same, to any degree or level relative to a control or a reference antibody. For example, the decrease in ADCC activity provided by the methods of the disclosure is at least or about a 1% to about a 100% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 9.8% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, at least or about a 100% decrease) relative to the level of a control or a reference antibody. In exemplary embodiments, the decrease provided by the methods of the disclosure is over about 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to the level of a control or a reference antibody. In exemplary embodiments, the level of ADCC activity of the antibody or composition comprising the same decreases by an amount falling within the range of about 5% to about 400%, relative to a control or a reference antibody. In exemplary embodiments, the level of ADCC activity of the antibody, or composition comprising the same decreases by: at least or about 1.5-fold, at least or about 2-fold, by at least or about 3-fold, at least or about 4-fold, or by at least or about 5-fold, relative to a control or a reference antibody. In exemplary embodiments, the level of ADCC activity of the antibody or composition comprising the same decreases by about 6-fold, about 7-fold, about 8-fold, about 9-fold, or about 10-fold, relative to a control or a reference antibody. In exemplary embodiments, the level of ADCC activity of the antibody or composition comprising the same decreases by an amount falling within the range of about 0.5-fold to about 8-fold, relative to a control or a reference antibody.

### Glycans

In exemplary embodiments, the methods disclosed herein comprises modulating the amount of glycans on an antibody including modulating: (a) galactosylated glycans; (b) afucosylated glycans; or (c) a combination thereof (e.g., galactosylated and afucosylated glycans) to increase or decrease ADCC activity of the antibody. In exemplary aspects, the methods disclosed herein comprises modulating the amount of glycans attached to the Fc domain at of an antibody including modulating: (a) galactosylated glycans; (b) afucosylated glycans (e.g., by way of modulating fucose); or (c) a combination thereof (e.g., galactosylated and afucosylated glycans) to increase or decrease ADCC activity of the antibody. In additional exemplary aspects, the methods disclosed herein comprises modulating the amount of glycans attached at the consensus N-glycosylation site in the CH2 domain of the Fc domain of an antibody including modulating: (a) galactosylated glycans; (b) afucosylated glycans (e.g., by way of modulating fucose); or (c) a combination thereof (e.g., galactosylated and afucosylated glycans) to increase or decrease ADCC activity of the antibody.

In exemplary aspects, the methods provided by the present disclosure relate to modulation of an IgG1 antibody composition wherein steps are taken to achieve a desired or predetermined or pre-selected level of glycoforms of the IgG1 antibody to achieve a desired or predetermined or pre-selected level of ADCC activity. In exemplary embodiments, the method comprises modulating (increasing or decreasing) the amount of galactosylated glycoforms of the IgG1 antibody to modulate (increase or decrease) the ADCC activity induced or stimulated by the antibody composition. In exemplary embodiments, the method comprises modulating (increasing or decreasing) the amount of glycoforms which are both galactosylated and afucosylated (i.e., galactosylated, afucosylated glycoforms) to modulate (increase or decrease) the ADCC activity induced or stimulated by the antibody composition. Without being bound to a particular theory, it is believed that the methods of the disclosure provide a means for tailor-made antibody compositions comprising specific amounts of particular glycoforms of a given antibody useful for achieving a particular level of ADCC activity. According, in some aspects, the methods disclosed herein comprises modulating the amount or percentage of galactosylated glycans, afucosylated glycans, or galactosylated, afucosylated glycans within an antibody composition.

In alternative aspects, the methods disclosed herein comprises modulating the amount of terminal β-galactose attached to a particular IgG1 molecule. For example, the method may comprise increasing the amount of terminal galactose on an IgG1 antibody (by, e.g., but not limited to, effectively changing the glycan from a G0 to a G1 or G2 species or from a G1 to a G2 species) to increase ADCC activity of the IgG1 antibody. Alternatively, the method may comprise decreasing the amount of terminal galactose (by, e.g., but not limited to, changing the glycan from a G2 to a G1 or G0 species or from a G1 to a G0 species) to decrease ADCC activity of the IgG1 antibody. In some embodiments, the methods comprise modulating the amount of terminal β-galactose of a glycosylated and afucosylated IgG1 antibody (such as an anti-HER2 antibody, an anti-TNFα, or an anti-CD20 antibody, including trastuzumab, infliximab or rituximab) to modulate ADCC activity of the IgG1 antibody. In some aspects, the methods comprise increasing the amount of terminal galactose, (by, e.g., effectively changing the glycan from a G0 to a G1 or G2 species or from a G1 to a G2 species) to increase the ADCC activity of the glycosylated and afucosylated IgG1 antibody, such as an anti-HER2 antibody, an anti-TNFα, or an anti-CD20 antibody, including trastuzumab, infliximab or rituximab. Alternatively, the methods herein may comprise decreasing the amount of terminal galactose, (by, e.g., but not limited to, changing the glycan from a G2 to a G1 or G0 species or from a G1 to a G0 species) to decrease the ADCC activity of the glycosylated and afucosylated IgG1 antibody, such as an anti-HER2 antibody, an anti-TNFα, or an anti-CD20 antibody, including trastuzumab, infliximab or rituximab.

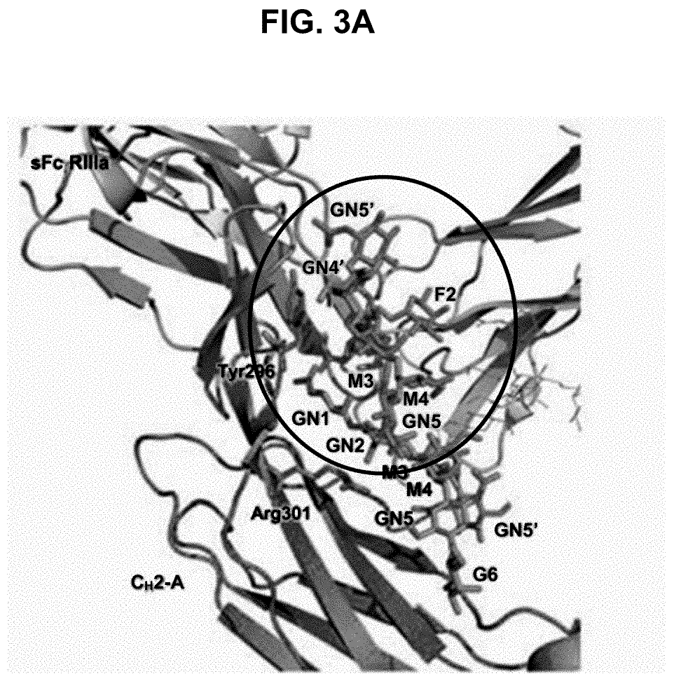

The term “glycan”, “glycans”, “glycoform” or “glycoforms” refers to oligomers of monosaccharide species that are connected by various glycosidic bonds. Examples of monosaccharides commonly found in mammalian N-linked glycans include hexose (Hex), glucose (Glc), galactose (Gal), mannose (Man) and N-acetylglucosamine (GlcNAc). The major N-glycan species found on recombinant IgG1 antibodies include fucose, galactose, mannose, sialic acid and GlcNAc, as depicted in FIG. 1. The glycan oligosaccharide structures are linked to the consensus N-glycosylation site in the CH2 domain and are generally composed of a core heptasaccharide with outer arms constructed by variable addition of fucose, N-acetylglucosamine (GlcNAc), galactose, sialic acid (SA), and bisecting N-GlcNAc. The representative oligosaccharide structures may be abbreviated as follows: A2G0F, A2G1F, A2G2F, A2G0, A2G1, A2G2 referring to the core GlcNAc and mannose oligosaccharide structure having zero, one or two terminal β-galactose moieties, with or without core fucose (F) attached respectively. Alternatively, abbreviations G0F, G1F, G2F, G0, G1 and G2 can be used, as shown in FIG. 2. Within G1, two additional structures, abbreviated G1a and G1b, may be present with G1a or G1b referring to whether the terminal galactose group is attached to either the 6-arm or the 3-arm of the core structure. When sialic acid is present, these abbreviations contain a “S” such that, for example, G2FS2 refers to a glycan having two galactose, a fucose and two sialic acid groups. Additional glycans linked to IgG1 antibodies may also exist including high mannose (HM) structures, which are formed by the incorporation of additional mannose groups, including the high mannose species “M9” and “A2G1S1M5” as shown in FIG. 1. As used herein, the term “glycan” or “glycoform” refers to any of the oligomers of monosaccharide species described herein or any other oligomers of monosaccharaide species linked to an antibody or an IgG1 antibody.

The terms “terminal β-galactose, “galactosylated glycans” or “G1, G1a, G1b and/or G2 galactosylated species” refers to a glycan comprising one (e.g., G1, including G1a and G1b) or two galactose (e.g., G2) molecules linked to an IgG1 antibody at the consensus N-glycosylation site in the CH2 domain through the N-acetylglucosamine moieties that attach to the core mannose structure. Exemplary glycans comprising “terminal β-galactose”, “galactosylated glycans” or A2G1F, A2G2F for fucose-containing glycans, as well as afucosylated forms A2G1 (including A2G1a and A2G1b) and A2G2 (or G1 and G2) are depicted in FIG. 2. In some embodiments, the galactosylated glycan is a hybrid glycan comprising a high mannose arm and a galactose-containing arm, as well as single-arm glycans exemplified by A1G1M5 and A1G1 respectively in FIG. 2.

The term “core fucose” or “fucosylated species” refers to a glycan comprising a fucose molecule (alpha 1-6) linked to an IgG1 antibody at the consensus N-glycosylation site in the CH2 domain through the n-acetylglucoseamine moieties that attach to the core mannose structure. Exemplary glycan comprising “core fucose” or “fucosylated species” are depicted in FIGS. 1 and 2. In some embodiments, antibodies containing core fucose and/or a fucosylated species may or may not contain other glycans including terminal β-galactose and/or high mannose.

The term “afucosylated”, “afucosylated glycans” or “afucosylation” refers to the removal or lack of core fucose in an antibody. Exemplary afucosylated antibody species are depicted in FIG. 2. In some embodiments, antibodies lacking core fucose may or may not contain other glycans including terminal β-galactose and/or high mannose. Afucosylated glycoforms include, but are not limited to, A1G0, A1G1a, A2G0, A2G1a, A2G1b, A2G2, and A1G1M5. See, e.g., Reusch and Tejada, Glycobiology 25(12): 1325-1334 (2015).

The term “high mannose”, “high mannose glycans” or “HM” refers to a glycan comprising more than 3 mannose molecules linked to an IgG1 antibody at the consensus N-glycosylation site in the CH2 domain. Exemplary high mannose antibodies are depicted in FIGS. 1 and 2. High mannose glycans encompass glycans comprising 5, 6, 7, 8, or 9 mannose residues, abbreviated as Man5, Man6, Man7, Man8, and Man9, or M5, M6, M7, M8, and M9, respectively.

The phrase “a glycosylated and afucosylated IgG1 antibody composition” or “afucosylated composition” used herein refers to an IgG1 antibody composition wherein antibodies within the composition contain a glycan oligosaccharide structure linked to the consensus N-glycosylation site in the CH2 domain. In preferred embodiments, the composition comprises antibodies comprising heptasaccharide cores wherein at least about 0.5% are afucosylated, or greater than about 0.5% are afucosylated, or between about 0.5% and 100% are afucosylated (or alternatively having 99.5% core fucose or less than 99.5% core fucose or having core fucose falling in the range between 0% and 99.5%).

### Modulating Amounts of Glycans

In exemplary embodiments, the methods described herein comprise modulating (i.e. increasing or decreasing) the amount or percentage of glycans, including, e.g., G1, G1a, G1b and/or G2 galactosylated species, of an IgG1 antibody composition.

The term “amount” when referring the amount of a glycan (including, e.g., (1) the amount of terminal β-galactose, (2) the amount of G1, G1a, G1b and/or G2 galactosylated species, (3) the amount of core fucose, (4) the amount of afucosylated species, or (5) the amount of galactosylated and afucosylated glycans) refers to a relative amount or percentage of a particular glycan compared to the total amount of glycans in the sample or the glycoprotein. For example, the amount of (1) terminal β-galactose, (2) G1, G1a, G1b and/or G2 galactosylated species, and/or (3) core fucose/afucosylated species, is denoted as a percentage calculated as the amount of species with terminal β-galactose, including G1, G1a, G1b and/or G2 galactosylated species or core fucose/afucosylated species, divided by the total amount of all glycans species in the sample or the glycoprotein. Methods for measuring and determining the amount or relative percentage of a glycan (including, e.g., G1, G1a, G1b and/or G2 galactosylated species, core fucose, afucosylated species, etc.) are well known in the art and include Hydrophilic Interaction Liquid Chromatography (HILIC)) as described in the Examples. See also, Pace et al., Characterizing the Effect of Multiple Fc Glycan Attributes on the Effector Functions and FccRIIIa Receptor Binding Activity of an IgG1 Antibody, Biotechnol. Prog., 2016, Vol. 32, No. 5 pages 1181-1192; and Shah, B. et al. LC-MS/MS Peptide Mapping with Automated Data Processing for Routine Profiling of N-Glycans in Immunoglobulins J. Am. Soc. Mass Spectrom. (2014) 25: 999, herein each incorporated by reference for all purposes. In some embodiments, amount can be determined or calculated as mole percent incorporation.

“Modulating”, as used herein, means to change by decreasing or increasing, and accordingly, in exemplary aspects, the method comprises increasing the amount of glycans of the antibody, while in alternative aspects, the method comprises decreasing the amount of glycans of the antibodies within a composition. In exemplary aspects, the methods of the present disclosure comprise increasing the glycans (e.g., galactosylated glycans, G1, G1a, G1b and/or G2 galactosylated species, afucosylated glycans, core fucose, or a combination thereof (e.g., galactosylated and afucosylated species)) of the antibodies within a composition, to any degree or level relative to a control or a reference antibody composition. In exemplary instances, the method comprises increasing the glycans (including, e.g., terminal β-galactose of glycosylated and afucosylated IgG1 antibodies within a composition; such as an anti-HER2 antibody composition, an anti-TNFα antibody composition, or an anti-CD20 antibody composition, including trastuzumab, infliximab or rituximab) by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or reference antibody composition. In exemplary embodiments, the method comprises increasing the glycans by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody composition. In exemplary embodiments, the level glycans the antibody composition increases falls within the range of about 5% to about 400%, relative to a control or a reference antibody composition. In exemplary embodiments, the method comprises increasing the glycans by: at least or about 1.5-fold, at least or about 2-fold, at least or about 3-fold, at least or about 4-fold or at least or about 5-fold, relative to a control or a reference antibody composition. In exemplary embodiments, the method comprises increasing the glycans by about 6-fold, about 7-fold, about 8-fold, about 9-fold, or about 10-fold, relative to a control or a reference antibody composition. In exemplary embodiments, the method comprises increasing the glycans by an amount falling within the range of about 0.5-fold to about 8-fold, relative to a control or a reference antibody composition.

In exemplary aspects, the methods of the present disclosure comprise decreasing the glycans (e.g., galactosylated glycans, G1, G1a, G1b and/or G2 galactosylated species, afucosylated glycans, core fucose, or a combination thereof (e.g., galactosylated, afucosylated glycans)) of the antibody composition, to any degree or level relative to a control or a reference antibody composition. In exemplary instances, the method comprises decreasing the glycans (including, e.g., terminal β-galactose of glycosylated and afucosylated IgG1 antibodies in a composition; such as an anti-HER2 antibody composition, an anti-TNFα antibody composition, or an anti-CD20 antibody composition, including trastuzumab, infliximab or rituximab) by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or a reference antibody composition. In exemplary embodiments, the method comprises decreasing the glycans by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody composition. In exemplary embodiments, the glycans of the antibody composition decreases by an amount falling within the range of about 5% to about 400%, relative to a control or a reference antibody composition. In exemplary embodiments, the method comprises decreasing the glycans by: at least or about 1.5-fold, at least or about 2-fold, at least or about 3-fold, at least or about 4-fold or at least or about 5-fold, relative to a control or a reference antibody composition. In exemplary embodiments, the method comprises decreasing the glycans by about 6-fold, about 7-fold, about 8-fold, about 9-fold, or about 10-fold, relative to a control or a reference antibody composition. In exemplary embodiments, the method comprises decreasing the glycans by an amount falling within the range of about 0.5-fold to about 8-fold, relative to a control or a reference antibody composition.

In exemplary aspects, the methods of the present disclosure comprise modulating (i.e. increasing or decreasing) the amount of galactosylated glycans or G1, G1a, G1b and/or G2 galactosylated species of the antibody composition to a total amount of at least or about 0.5%, at least or about 1%, at least or about 2%, at least or about 3%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97% or at least or about 98% or increased or decreased to a total amount in the range of at least or about 0.5% to 98% or increased or decreased to a total amount in the range of 0% to 100%.

In exemplary aspects, the methods of the present disclosure comprise modulating (i.e. increasing or decreasing) the amount of galactosylated glycans or G1, G1a, G1b and/or G2 galactosylated species and afucosylated glycans of the antibody composition, wherein the a total amount of galactosylated glycans or G1, G1a, G1b and/or G2 galactosylated species is at least or about 0.5%, at least or about 1%, at least or about 2%, at least or about 3%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97% or at least or about 98% or increased or decreased to a total amount in the range of at least or about 0.5% to 98% or increased or decreased to a total amount in the range of 0% to 100%; and the total amount of afucosylated glycans is at least about 0.5% or greater than about 0.5%, or at least or about 3%, at least or about 4%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97% or at least or about 98%, or at least of about 99%, or increased or decreased to a total amount in the range of about 0.5% to 100%, a total amount in the range of about 3% to 100%, a total amount in the range of about 5% to 100%, or a total amount in the range of about 8% to 100%.

In exemplary embodiments, the methods of the present disclosure comprise modulating the amount of galactosylated glycans, including, e.g., terminal β-galactose or G1, G1a, G1b and/or G2 galactosylated species, of the antibody composition to modulate its ADCC activity. In exemplary aspects, the method comprises increasing the amount of galactosylated glycans, including, e.g., terminal β-galactose or G1, G1a, G1b and/or G2 galactosylated species, of the antibody composition to increase its ADCC activity. In exemplary aspects, the method comprises decreasing the amount of galactosylated glycans including, e.g., terminal (3-galactose or G1, G1a, G1b and/or G2 galactosylated species, of the antibody composition to decrease its ADCC activity. In exemplary aspects, the method comprises increasing the amount of galactosylated glycans, including, e.g., terminal β-galactose or G1, G1a, G1b and/or G2 galactosylated species, of an afucosylated IgG1 antibody composition to increase its ADCC activity. In exemplary aspects, the method comprises decreasing the amount of galactosylated glycans including, e.g., terminal β-galactose or G1, G1a, G1b and/or G2 galactosylated species, of an afucosylated IgG1 antibody composition to decrease its ADCC activity.

In exemplary embodiments, the methods of the present disclosure comprise modulating the amount of galactosylated glycans, including, e.g., terminal β-galactose or G1, G1a, G1b and/or G2 galactosylated species, and afucosylated glycans or the amount of core fucose of the antibody composition to modulate its ADCC activity. In exemplary aspects, the method comprises increasing ADCC activity of an IgG1 antibody composition by both (1) increasing the amount of galactosylated glycans, including, e.g., terminal β-galactose or G1, G1a, G1b and/or G2 galactosylated species, and (2) increasing afucosylated glycans or decreasing the amount of core fucose. In exemplary aspects, the method comprises decreasing ADCC activity of an IgG1 antibody composition by both (1) decreasing the amount of galactosylated glycans, including, e.g., terminal β-galactose or G1, G1a, G1b and/or G2 galactosylated species, and (2) decreasing the amount of afucosylated glycans or increasing the amount of core fucose. In some embodiments, the IgG1 antibody is an anti-HER2 antibody, an anti-TNFα antibody, or an anti-CD20 antibody, including trastuzumab, infliximab or rituximab.

### Methods of Engineering ADCC Activity of an Antibody

The methods provided herein also include methods of matching the ADCC activity of a first, reference IgG1 antibody composition and the ADCC activity of a second antibody composition by modulating the amount of glycans (e.g., galactosylated glycans, terminal β-galactose, G1, G1a, G1b and/or G2 galactosylated species, afucosylated glycans, core fucose, or a combination thereof (e.g., galactosylated and afucosylated glycans)) in the second antibody composition to match the ADCC activity of the first, reference IgG1 antibody composition.

For example, in some exemplary embodiments the methods of the present disclosure comprise matching the ADCC of a reference glycosylated and afucosylated IgG1 antibody composition by (1) determining the ADCC activity of a reference glycosylated and afucosylated IgG1 antibody composition; (2) determining the ADCC activity of a second antibody composition wherein the antibody has the same antibody sequence as the reference antibody; and (3) changing the ADCC activity of the second antibody composition by increasing or decreasing the amount of terminal β-galactose (including, e.g., the amount of G1, G1a, G1b and/or G2 galactosylated species) in the glycan species at the consensus glycosylation site of antibodies in the second composition, wherein the ADCC activity of the second antibody composition after increasing or decreasing the amount of terminal β-galactose is the same as the reference IgG1 antibody composition or within about 10%, about 15%, about 20%, about 25%, about 30%, about 35%, about 40%, about 45% or about 50% of the reference IgG1 antibody composition or within about 1% to about 50% of the reference IgG1 antibody composition. In exemplary aspects, an increase of about 1% terminal β-galactose increases ADCC activity by about 20% to about 30%. In exemplary aspects, a decrease of about 1% terminal β-galactose decreases ADCC activity by about 20% to about 30%. In exemplary aspects, the method comprises modulating the amount or percentage of galactosylated and afucosylated glycans of the second antibody composition to modulate ADCC activity of the antibody composition to match the ADCC activity of the reference glycosylated and afucosylated IgG1 antibody composition. In exemplary aspects, the method comprises increasing the ADCC activity of the second antibody composition by increasing the amount or percentage of galactosylated and afucosylated glycans of the second antibody composition to match the ADCC activity of the reference glycosylated and afucosylated IgG1 antibody composition. In exemplary aspects, the method comprises decreasing the ADCC activity of the second antibody composition by decreasing the amount or percentage of galactosylated and afucosylated glycans of the second antibody composition to match the ADCC activity of the reference glycosylated and afucosylated IgG1 antibody composition. In some embodiments, step 1 of the method (i.e. “determining the ADCC activity of a reference glycosylated and afucosylated IgG1 antibody composition”) occurs before, after or at the same time as steps 2 and/or steps 3 of the method.

In addition to methods of matching the ADCC of a reference antibody composition, the methods provided herein also contemplate methods of engineering an antibody composition with a specific ADCC activity by modulating the amount of glycans (e.g., galactosylated glycans, terminal β-galactose, G1, G1a, G1b and/or G2 galactosylated species, afucosylated glycans, core fucose, or a combination thereof (e.g., galactosylated, afucosylated glycans) of the antibody composition to achieve a target, desired or pre-selected ADCC activity.

For example, in some exemplary embodiments of the methods of the present disclosure, the method comprises engineering a specific target ADCC activity in an antibody composition by: (1) determining the ADCC activity of a glycosylated and afucosylated IgG1 antibody composition; (2) determining a target ADCC activity; and (3) increasing or decreasing the ADCC activity of the IgG1 antibody composition by increasing or decreasing the amount of terminal β-galactose (including, e.g., G1, G1a, G1b and/or G2 galactosylated species) in the glycan species at the consensus glycosylation site, wherein the ADCC activity of the antibody composition after increasing or decreasing the amount of terminal β-galactose is the same as the target ADCC activity or within about 10%, about 15%, about 20%, about 25%, about 30%, about 35%, about 40%, about 45% or about 50% of the target ADCC activity or within about 1% to about 50% of the target ADCC activity. In exemplary aspects, an increase of about 1% terminal β-galactose increases ADCC activity by about 2%. In exemplary aspects, a decrease of about 1% terminal β-galactose decreases ADCC activity by about 2%. In exemplary aspects, the method comprises modulating the amount or percentage of galactosylated and afucosylated glycans of the IgG1 antibody composition to match the target ADCC activity. In exemplary aspects, the method comprises increasing ADCC activity of the IgG1 antibody composition by increasing the amount or percentage of galactosylated and afucosylated glycans of the IgG1 antibody composition to match the target ADCC activity. In exemplary aspects, the method comprises decreasing ADCC activity of the IgG1 antibody composition by decreasing the amount of galactosylated and afucosylated glycans of the IgG1 antibody composition to match the target ADCC activity. In some embodiments, step 1 of the method (i.e. “determining the ADCC activity of a glycosylated and afucosylated IgG1 antibody composition”) occurs before, after or at the same time as steps 2 and/or steps 3 of the method.

### Methods of Modulating Glycans

Suitable methods of modulating glycans (such as galactosylated glycans (including, e.g., terminal β-galactose or G1, G1a, G1b and/or G2 galactosylated species), and/or afucosylated glycans) on glycoproteins, including antibodies, are known in the art. For example, see Zhang et al., Drug Discovery Today 21(5): 2016), which reviews the effects of cell culture conditions on glycosylation. See also the methods described in the Examples.

Thus, in some aspects, glycosylation-competent cells—which can be used to recombinantly produce a glycoprotein, including antibodies—are cultured under particular conditions to achieve the desired level of glycans in antibody composition produced using the cells. For example, International Patent Publication Nos. WO2013/114164; WO 2013/114245; WO 2013/114167; WO 2015128793; and WO 2016/089919 each teach recombinant cell culturing techniques useful to modulate glycans, such as galactosylated glycans (including, e.g., terminal β-galactose or G1, G1a, G1b and/or G2 galactosylated species), afucosylated glycans or glycans containing core fucose, including: methods of obtaining glycoproteins having increased percentage of total afucosylated glycans (WO2013/114164); methods of obtaining glycoproteins having increased percentage of Man5 glycans and/or afucosylated glycans (WO 2013/114245); methods of obtaining glycoproteins having specific amounts of high mannose glycans, afucosylated glycans and G0F glycans (WO 2013/114167); methods of obtaining glycoproteins having high mannose glycan and reduced galactosylation and/or high galactosylated glycans (WO 2015128793); and methods of manipulating the fucosylated glycan content on a recombinant protein (WO2016/089919). The cell culture techniques described by WO2013/114164; WO 2013/114245; WO 2013/114167; WO 2015128793; and WO 2016/089919 include modifying one or more cell culture parameters such as temperature, pH, culturing cells with manganese ion or salts thereof (e.g., 0.35 μM to about 20 μM Manganese) and/or culturing cells with copper (e.g., 10 to 100) and manganese (e.g., 50 to 1000 nM).

Additionally, International Patent Publication No. WO2015/140700 teaches culturing cells with betaine to increase afucosylated glycans, and further teaches culturing cells with manganese, galactose and betaine for obtaining target values of mannosylated, galactosylated and afucosylated glycans. Similarly, Konno et al., Cytotechnology 64: 249-3+6 (2012) teaches that fucose content of antibodies can be controlled by culture medium osmolality. International Patent Publication No. WO2017/079165 describes culturing genetically modified host cells having no GMD or FX with fucose to produce afucosylated and fucosylated forms of the protein. International Patent Publication No. WO2017/134667 describes manipulating glycan content by culturing cells with nicotinamide and fucose at a concentration of at least 1 mM. Sha et al., TIBs 34(10): 835-846 (2016) also reviews several methods of modulating glycans, including, for example, using a combination of uridine, manganese, and galactose to increase galactosylation levels on antibodies, and using mannose as a carbon source to increase high mannose glycoforms. Additionally, McCracken et al., Biotechnol. Prog. 30(3): 547-553 (2014) teaches methods of controlling galactosylated glycoform distribution in cell culture, involving cell culture medium comprising particular asparagine concentrations, ammonium levels, and pH to influence the amounts of G0F, G1F, and G2F.

Accordingly, the methods of the present disclosure, in exemplary aspects, comprises adopting one or more of the practices and/or conditions taught in any one or more of the above references or other reference described herein, in order to modulate the amounts of the galactosylated glycans (including, e.g., terminal β-galactose or G1, G1a, G1b and/or G2 galactosylated species), and/or afucosylated glycans or glycans containing core fucose within an antibody composition. In exemplary aspects, the method comprises culturing glycosylation-competent cells expressing the antibody in a cell culture medium under conditions which modulate the level(s) of the galactosylated glycans (including, e.g., terminal β-galactose or G1, G1a, G1b and/or G2 galactosylated species), and/or afucosylated glycans or glycans containing core fucose.

In the methods described herein comprising maintaining or culturing cells in cell culture, the cell culture may be maintained according to any set of conditions suitable for a recombinant glycosylated protein or antibody production. For example, in some aspects, the cell culture is maintained at a particular pH, temperature, cell density, culture volume, dissolved oxygen level, pressure, osmolality, and the like suitable for recombinant glycosylated protein or antibody production. In exemplary aspects, the cell culture prior to inoculation is shaken (e.g., at 70 rpm) at 5% CO₂ under standard humidified conditions in a CO₂ incubator.

In exemplary aspects, the methods of the disclosure comprise maintaining the glycosylation-competent cells in a cell culture medium at a pH, temperature, osmolality, and dissolved oxygen level suitable for recombinant glycosylated protein or antibody production, as well-known in the art. In exemplary aspects, the cell culture is maintained in a medium suitable for cell growth and/or is provided with one or more feeding media according to any suitable feeding schedule as well-known in the art.

In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4: 217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases.

In additional or alternative aspects, the glycosylation-competent cells which recombinantly produce the antibody are genetically modified in a way to modulate the glycans (such as the galactosylated glycans (including, e.g., terminal β-galactose or G1, G1a, G1b and/or G2 galactosylated species), and/or afucosylated glycans or glycans containing core fucose) of the antibodies produced by the cell. In exemplary aspects, the glycosylation-competent cells are genetically modified to alter activity of an enzyme of the de novo pathway or the salvage pathway. Optionally, the glycosylation-competent cells are genetically modified to knock-out a gene encoding GDP-keto-6-deoxymannonse-3,5-epimerase, 4-reductase. In exemplary embodiments, the glycosylation-competent cells are genetically modified to alter the activity of an enzyme of the de novo pathway or the salvage pathway. These two pathways of fucose metabolism are well-known in the art and shown in FIG. 5D. In exemplary embodiments, the glycosylation-competent cells are genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are genetically modified to knock-out a gene encoding FX. In exemplary embodiments, the glycosylation-competent cells are genetically modified to alter the activity β(1,4)-N-acetylglucosaminyltransferase III (GNTIII) or GDP-6-deoxy-D-lyxo-4-hexulose reductase (RMD). In exemplary aspects, the glycosylation-competent cells are genetically modified to overexpress GNTIII or RMD. In exemplary embodiments, the glycosylation-competent cells are genetically modified to have altered beta-galactosyltransferase activity.

Several ways are known in the art for reducing or abolishing fucosylation of Fc-containing molecules, e.g., antibodies. These include recombinant expression in certain mammalian cell lines including a FUT8 knockout cell line, variant CHO line Lec13, rat hybridoma cell line YB2/0, a cell line comprising a small interfering RNA specifically against the FUT8 gene, and a cell line coexpressing β-1,4-N-acetylglucosaminyltransferase III and Golgi α-mannosidase II. Alternatively, the Fc-containing molecule may be expressed in a non-mammalian cell such as a plant cell, yeast, or prokaryotic cell, e.g., E. coli.

In exemplary aspects, targeted glycan amounts are achieved through post-production chemical or enzyme treatment of the antibody composition. In exemplary aspects, the method of the present disclosure comprises treating the antibody composition with a chemical or enzyme after the antibodies are recombinantly produced. In exemplary aspects, the chemical or enzyme is selected from the group consisting of EndoS; Endo-S2; Endo-D; Endo-M; endoLL; α-fucosidase; β-(1-4)-Galactosidase; Endo-H; Endo F1; Endo F2; Endo F3; β-1,4-galactosyltransferase; kifunensine, and PNGase F. In exemplary aspects, the chemical or enzyme is incubated with the antibody composition at various times to generate antibodies having different amounts of glycans. In some aspects, the antibody composition is incubated with β-1,4-galactosyltransferase (GalTase) as described in the Examples. In some additional aspects, antibodies having different levels of galactose can be generated by incubating the antibody composition with β-1,4-galactosyltransferase for a set period of time, including, but not limited to, about 10 minutes, about 20 minutes, about 30 minutes, about 1 hour, about 2 hours, about 4 hours, about 9 hours or for a period of time falling in the range between about 10 minutes and about 9 hours.

### Methods of Measuring Glycans

Various methods are known in the art for assessing glycoforms present in a glycoprotein-containing composition, including antibody compositions, or for determining, detecting or measuring a glycoform profile of a particular sample comprising glycoproteins. Suitable methods include, but are not limited to, Hydrophilic Interaction Liquid Chromatography (HILIC), Liquid chromatography-tandem mass spectrometry (LC-MS), positive ion MALDI-TOF analysis, negative ion MALDI-TOF analysis, HPLC, weak anion exchange (WAX) chromatography, normal phase chromatography (NP-HPLC), exoglycosidase digestion, Bio-Gel P-4 chromatography, anion-exchange chromatography and one-dimensional n.m.r. spectroscopy, and combinations thereof. See, e.g., Pace et al., Biotechnol. Prog., 2016, Vol. 32, No. 5 pages 1181-1192; Shah, B. et al. J. Am. Soc. Mass Spectrom. (2014) 25: 999; Mattu et al., JBC 273: 2260-2272 (1998); Field et al., Biochem J 299(Pt 1): 261-275 (1994); Yoo et al., MAbs 2(3): 320-334 (2010) Wuhrer M. et al., Journal of Chromatography B, 2005, Vol. 825, Issue 2, pages 124-133; Ruhaak L. R., Anal Bioanal Chem, 2010, Vol. 397:3457-3481; Kurogochi et al., PLOS One 10(7): e0132848 (2015); Thomann et al., PLOS One 10(8): e0134949. (2015); Pace et al., Biotechnol. Prog. 32(5): 1181-1192 (2016); and Geoffrey, R. G. et. al. Analytical Biochemistry 1996, Vol. 240, pages 210-226. Also, the examples set forth herein describe a suitable method for assessing glycoforms present in a glycoprotein containing composition such as an antibody composition.

### Control

As described herein, some of the methods of the disclosure recite a modulation (e.g., an increase or decrease) effected by such methods that are relative to a “control” or “reference” antibody composition. In exemplary aspects, with regard to ADCC activity or amount of glycans, the “control” is the level of ADCC activity and/or amount of glycans of the antibody composition (e.g., a reference antibody composition) prior to any experimental intervention directed at modulating ADCC activity and/or modulating glycan profile, such as the level of ADCC activity and/or amount of glycans of the antibody composition (e.g., a reference antibody composition) when first measured or determined. In certain aspects, a “control” or “reference” antibody composition can be an antibody composition that has undergone significant experimental intervention directed at modulating ADCC activity and/or modulating glycan profile but where additional modulation of ADCC activity and/or glycan profile is desired. In these instances, the “control” is the level of ADCC activity and/or amount of glycans of the antibody composition (e.g., a reference antibody composition) prior to any additional experimental intervention directed at further modulating ADCC activity and/or further modulating glycan profile.

### Antibody, Fragments, and Protein Products

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes”, Immunobiology: The Immune System in Health and Disease, 4th ed. Elsevier Science Ltd./Garland Publishing, (1999).

The term “antibody fragment” or “antibody fragment thereof” refers to a portion of an intact antibody. An “antigen-binding fragment” or “antigen-binding fragment thereof” refers to a portion of an intact antibody that binds to an antigen. An antigen-binding fragment can contain the antigenic determining variable regions of an intact antibody. Examples of antibody fragments antigen-binding fragment include, but are not limited to Fab, Fab′, F(ab′)2, and Fv fragments, linear antibodies, scFvs, and single chain antibodies.

The term “IgG” as used herein refers to a polypeptide belonging to the class of antibodies that are substantially encoded by a recognized immunoglobulin gamma gene. In humans, this class comprises IgG1, IgG2, IgG3, and IgG4. In mice, this class comprises IgG1, IgG2a, IgG2b, and IgG3. The sequences of the heavy chains of human IgG1, IgG2, IgG3 and IgG4 can be found in many sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively. In preferred embodiments, the methods and antibodies disclosed herein relate to IgG1 antibodies. In some other preferred embodiments, the methods and antibodies disclosed herein relate to human IgG1 antibodies.

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. MoI. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred. See, e.g., Chothia and Lesk, J. Mol. Biol., 1987, 196: 901; Chothia et al., Nature, 1989, 342: 877; Martin and Thornton, J. Mol. Biol, 1996, 263: 800; Antibodies: A Laboratory Manual, Cold Spring Harbor Laboratory, eds. Harlow et al., 1988, each herein incorporated by reference.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding site.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.).

The terms “Fc domain,” “Fc Region,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor. For example, an Fc domain contains the entire second constant domain CH2 (residues at EU positions 231-340 of human IgG1) and the third constant domain CH3 (residues at EU positions 341-447 of human IgG1).

Fc can refer to this region in isolation, or this region in the context of an antibody, or antibody fragment. Polymorphisms have been observed at a number of positions in Fc domains, including but not limited to EU positions 270, 272, 312, 315, 356, and 358. Thus, a “wild type IgG Fc domain” or “WT IgG Fc domain” refers to any naturally occurring IgG Fc region (i.e., any allele). Myriad Fc mutants, Fc fragments, Fc variants, and Fc derivatives are described, e.g., in U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,122,637; 7,183,387; 7,332,581; 7,335,742; 7,371,826; 6,821,505; 6,180,377; 7,317,091; 7,355,008; U.S. Patent publication 2004/0002587; and PCT Publication Nos. WO 99/058572, WO 2011/069164 and WO 2012/006635.

The Fc region generally determines the antibody effector function that will ensue after antigen binding. It can recruit molecules in the innate immune system, such as C1q, as well as cytotoxic and antigen-presenting cells via binding interactions with Fcγ receptors. The IgG Fc region contains two conserved N-glycosylation sites at Asn297, one on each heavy chain (see P. M. Rudd. Glycosylation and the immune system. Science, 291 (2001), pp. 2370-2376). Variations in the structure glycans at the consensus N-glycosylation site results in subtle changes in structure that influence the interaction of IgG with the immune system. For example, Fc region glycans can directly influence the affinity of IgGs to Fey receptors, either by changing the conformation of the Fc region (see S. Krapp, et al. Structural analysis of human IgG-Fc glycoforms reveals correlation between glycosylation and structural integrity J. Mol. Biol., 325 (2003); 979-98931; Y. Mimura, et al. Role of oligosaccharide residues of IgG1-Fc in Fc RIIb binding J. Biol. Chem., 276 (2001), 45539-45547) or through glycan-glycan interactions (see C. Ferrara, et al. Unique carbohydrate-carbohydrate interactions are required for high affinity binding between Fc(RIII and antibodies lacking core fucose. Proc. Natl. Acad. Sci. U.S.A., 108 (2011), 12669-12674), thus strongly influencing their ability to recruit immune effector cells. See also, Zhang et al. Challenges of glycosylation analysis and control: an integrated approach to producing optimal and consistent therapeutic drugs. Drug Discovery Today, (21) 5 (2016) 740-765.

The term “monoclonal antibody” (mAb) as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target antigen (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dübel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

The monoclonal antibodies described in the present invention include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody” includes antibodies having antibody regions such as variable and constant regions or domains which correspond substantially to human germline immunoglobulin sequences known in the art, including, for example, those described by Kabat et al. (1991) (loc. cit.). The human antibodies, antibody constructs or binding domains of the invention may include amino acid residues not encoded by human germline immunoglobulin sequences (e.g., mutations introduced by random or site-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein also contemplates fully human antibodies, which include only non-artificially and/or genetically altered human sequences of antibodies as those can be derived by using technologies or systems such as the Xenomouse.

Advantageously, the methods described herein are not limited to specific antibodies or a particular type of antibody. In exemplary aspects, however, the antibody comprises an Fc domain, and in exemplary instances, the antibody is an IgG1 antibody. In exemplary embodiments, the antibody is an IgG1 antibody which has a particular antibody sequence. The term “antibody sequence” refers to the amino acid sequence of an antibody. The phrase used herein “having the same sequence as the reference antibody” refers to an antibody having an identical amino acid sequence to the amino acid sequence of a reference antibody's complementarity determining region (CDR), variable heavy chain (VH) and/or a variable light chain (VL). In preferred embodiments, an antibody “having the same sequence as a reference antibody” as used herein refers to an antibody having the same CDR, VH and VL amino acid sequences as a reference antibody's CDR, VH and VL sequences.

In exemplary aspects, the IgG1 antibody is an anti-EGFR antibody, e.g., an anti-HER2 monoclonal antibody. In exemplary aspects, the IgG1 antibody is trastuzumab, or a biosimilar thereof. The term trastuzumab refers to an IgG1 kappa humanized, monoclonal antibody that binds HER2/neu antigen (see CAS Number: 180288-69-1; DrugBank—DB00072; Kyoto Encyclopedia of Genes and Genomes (KEGG) entry D03257) comprising the VH and VL or VH-IgG1 and VL-IgG kappa sequences recited in Table 1 or set forth in SEQ ID Nos. 1-8, 21 or 22.

TABLE 1
Trastuzumab Amino Acid Sequences

| Description | Sequence | SEQ ID NO: |
|---|---|---|
| LC CDR1 | QDVNTA | 1 |
| LC CDR2 | SAS | 2 |
| LC CDR3 | QQHYTTPPT | 3 |
| HC CDR1 | GFNIKDTY | 4 |
| HC CDR2 | IYPTNGYT | 5 |
| HC CDR3 | SRWGGDGFYAMDY | 6 |
| VL | DIQMTQSPSSLSASVGDRVTITCRASQDVNTAVAWYQQKPGKAPKLL | |
| | IYSASFLYSGVPSRFSGSRSGTDFTLTISSLQPEDFATYYCQQHYTTPPT | 7 |
| | FGQGTKVEIK | |
| VH | EVQLVESGGGLVQPGGSLRLSCAASGFNIKDTYIHWVRQAPGKGLEW | 8 |
| | VARIYPTNGYTRYADSVKGRFTISADTSKNTAYLQMNSLRAEDTAVY | |
| | YCSRWGGDGFYAMDYWGQGTLVTVSS | |
| VL-IgG | DIQMTQSPSSLSASVGDRVTITCRASQDVNTAVAWYQQKPGKAPKLL | |
| Kappa | IYSASFLYSGVPSRFSGSRSGTDFTLTISSLQPEDFATYYCQQHYTTPPT | 21 |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| VH-IgG1 | EVQLVESGGGLVQPGGSLRLSCAASGFNIKDTYIHWVRQAPGKGLEW | 22 |
| | VARIYPTNGYTRYADSVKGRFTISADTSKNTAYLQMNSLRAEDTAVY | |
| | YCSRWGGDGFYAMDYWGQGTLVTVSSASTKGPSVFPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKVEPPKSCDKTHTCPPCPA | |
| | PELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVS | |
| | NKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQ | |
| | GNVFSCSVMHEALENHYTQKSLSLSPG | |

LC, light chain; HC, heavy chain; VL, variable light chain; VH, variable heavy chain.

In alternative aspects, the IgG1 antibody is an anti-CD20 antibody, e.g., an anti-CD20 monoclonal antibody. In alternative aspects, the IgG1 antibody is rituximab, or a biosimilar thereof. The term rituximab refers to an IgG1 kappa chimeric murine/human, monoclonal antibody that binds CD20 antigen (see CAS Number: 174722-31-7; DrugBank—DB00073; Kyoto Encyclopedia of Genes and Genomes (KEGG) entry D02994) comprising the VH and VL or comprising VH-IgG1 and VL-IgG kappa sequences recited in Table 2 or set forth in SEQ ID Nos. 11-18, 23 or 24.

TABLE 2
Rituximab Amino Acid Sequences

| Description | Sequence | SEQ ID NO: |
|---|---|---|
| LC CDR1 | RASSSVSYIH | 11 |
| LC CDR2 | ATSNLAS | 12 |
| LC CDR3 | QQWTSNPPT | 13 |
| HC CDR1 | SYNMH | 14 |
| HC CDR2 | AIYPGNGDTSYNQKFKG | 15 |
| HC CDR3 | STYYGGDWYFNV | 16 |
| VL | QIVLSQSPAILSASPGEKVTMTFCRASSSVSYIRWFQQKPGSSPKPWIYAT | 17 |
| | SNLASGVPVRFSGSGSGTSYSLTISRVEAEDAATYYCQQWTSNPPTFGG | |
| | GTKLEIK | |
| VH | QVQLQQPGAELVKPGASVKMSCKASGYTFTSYNMHWVKQTPGRGLE | 18 |
| | WIGAIYPGNGDTSYNQKFKGKATLTADKSSSTAYMQLSSLTSEDSAVY | |
| | YCARSTYYGGDWYFNVWGAGTTVTVSA | |
| VL-IgG | QIVLSQSPAILSASPGEKVTMTCRASSSVSYIHWFQQKPGSSPKPWIYAT | 23 |
| Kappa | SNLASGVPVRFSGSGSGTSYSLTISRVEAEDAATYYCQQWTSNPPTFGG | |
| | GTKLEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVT | |
| | HQGLSSPVTKSFNRGEC | |
| VH-IgG1 | QVQLQQPGAELVKPGASVKMSCKASGYTFTSYNMHWVKQTPGRGLE | 24 |
| | WIGAIYPGNGDTSYNQKFKGKATLTADKSSSTAYMQLSSLTSEDSAVY | |
| | YCARSTYYGGDWYFNVWGAGTTVTVSAASTKGPSVFPLAPSSKSTSG | |
| | GTAALGCLVKDYFPEPVTVSWNSGALTSGVHTFPAVLQSSGLYSLSSV | |
| | VTVPSSSLGTQTYICNVNHKPSNTKVDKKAEPKSCDKTHTCPPCPAPEL | |
| | LGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALP | |
| | APIEKTISKAKGQPREPQVYTLPPSRDELTKNQVSLTCLVKGFYPSDIAV | |
| | EWESNGQPENNYKTTPPVLDSDGSFFLYSKLTVDKSRWQQGNVFSCSV | |
| | MHEALHNHYTQKSLSLSPGK | |

LC, light chain; RC, heavy chain; VL, variable light chain; VH, variable heavy chain.

In exemplary aspects, the IgG1 antibody is an anti-TNFα antibody. In exemplary aspects, the IgG1 antibody is infliximab, or a biosimilar thereof. The term infliximab refers to an IgG1 kappa chimeric murine/human, monoclonal antibody that binds TNFα antigen (see CAS Number: 170277-31-3; DrugBank—DB00065; Kyoto Encyclopedia of Genes and Genomes (KEGG) entry D02598) comprising the VH and VL or comprising VH-IgG1 and VL-IgG kappa sequences recited in recited in Table 3 or set forth in SEQ ID Nos. 25-34.

TABLE 3
Infliximab Amino Acid Sequences

| Description | Sequence | SEQ ID NO: |
|---|---|---|
| LC CDR1 | FVGSSIH | 25 |
| LC CDR2 | KYASESM | 26 |
| LC CDR3 | QSHSW | 27 |
| HC CDR1 | IFSNHW | 28 |
| HC CDR2 | RSKSINSATH | 29 |
| HC CDR3 | NYYGSTY | 30 |
| VL | DILLTQSPAILSVSPGERVSFSCRASQFVGSSIHWYQQRTNGSPRLLIKY | 31 |
| | ASESMSGIPSRFSGSGSGTDFTLSINTVESEDIADYYCQQSHSWPFTFG | |
| | SGTNLEVK | |
| VH | EVKLEESGGGLVQPGGSMKLSCVASGFIFSNHWMNWVRQSPEKGLE | 32 |
| | WVAEIRSKSINSATHYAESVKGRFTISRDDSKSAVYLQMTDLRTEDTG | |
| | VYYCSRNYYGSTYDYWGQGTTLTVS | |
| VL-IgG | DILLTQSPAILSVSPGERVSFSCRASQFVGSSIHWYQQRTNGSPRLLIKY | 33 |
| Kappa | ASESMSGIPSRFSGSGSGTDFTLSINTVESEDIADYYCQQSHSWPFTFG | |
| | SGTNLEVKRTVAAPSVFIFPPSDEQLKSGTASVVCLLNNFYPREAKVQ | |
| | WKVDNALQSGNSQESVTEQDSKDSTYSLSSTLTLSKADYEKHKVYA | |
| | CEVTHQGLSSPVTKSFNRGEC | |
| VH-IgG1 | EVKLEESGGGLVQPGGSMKLSCVASGFIFSNHWMNWVRQSPEKGLE | 34 |
| | WVAEIRSKSINSATHYAESVKGRFTISRDDSKSAVYLQMTDLRTEDTG | |
| | VYYCSRNYYGSTYDYWGQGTTLTVSASTKGPSVFPLAPSSKSTSGGT | |
| | AALGCLVKDYFPEPVTVSWNSGALTSGVHTFPAVLQSSGLYSLSSVV | |
| | TVPSSSLGTQTYICNVNHKPSNTKVDKKAEPKSCDKTHTCPPCPAPEL | |
| | LGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNK | |
| | ALPAP1EKTISKAKGQPREPQVYTLPPSRDELTKNQVSLTCLVKGFYPS | |
| | DIAVEWESNGQPENNYKTTPPVLDSDGSFFLYSKLTVDKSRWQQGNV | |
| | FSCSVMHEALHNHYTQKSLSLSPGK | |

LC, light chain; HC, heavy chain; VL, variable light chain; VH, variable heavy chain.

### Additional Steps

The methods disclosed herein, in various aspects, comprise additional steps. For example, in some aspects, the methods comprise one or more upstream steps or downstream steps involved in producing, purifying, and formulating a recombinant protein, e.g., an antibody. In exemplary embodiments, the method comprises steps for generating host cells that express a recombinant glycosylated protein (e.g., antibody). The host cells, in some aspects, are prokaryotic host cells, e.g., E. coli or Bacillus subtilis, or the host cells, in some aspects, are eukaryotic host cells, e.g., yeast cells, filamentous fungi cells, protozoa cells, insect cells, or mammalian cells (e.g., CHO cells). Such host cells are described in the art. See, e.g., Frenzel, et al., Front Immunol 4: 217 (2013) and herein under “Cells.” For example, the methods comprise, in some instances, introducing into host cells a vector comprising a nucleic acid comprising a nucleotide sequence encoding the recombinant protein, or a polypeptide chain thereof

In exemplary embodiments, the methods disclosed herein comprise steps for isolating and/or purifying the recombinant protein (e.g., recombinant antibody) from the culture. In exemplary aspects, the method comprises one or more chromatography steps including, but not limited to, e.g., affinity chromatography (e.g., protein A affinity chromatography), ion exchange chromatography, and/or hydrophobic interaction chromatography. In exemplary aspects, the method comprises steps for producing crystalline biomolecules from a solution comprising the recombinant proteins.

The methods of the disclosure, in various aspects, comprise one or more steps for preparing a composition, including, in some aspects, a pharmaceutical composition, comprising the purified recombinant protein. Such compositions are discussed below.

### Compositions

Provided herein are also compositions comprising recombinant glycosylated proteins and antibodies produced by the methods described herein. In exemplary embodiments, the antibody compositions are prepared by methods which modulate the amount of glycans (e.g., galactosylated glycans, terminal β-galactose, G1, G1a, G1b and/or G2 galactosylated species, afucosylated glycans, core fucose, or a combination thereof). In exemplary aspects, the antibody is an IgG1 antibody. Accordingly, antibody compositions are provided herein, including glycosylated and afucosylated IgG1 antibodies (such as an anti-HER2 antibody, an anti-TNFα, or an anti-CD20 antibody, including trastuzumab, infliximab or rituximab) having increased or decreased ADCC activity, wherein the glycosylated and afucosylated IgG1 antibodies (such as an anti HER2 antibody, an anti-TNFα, or an anti-CD20 antibody, including trastuzumab, infliximab or rituximab) have been engineered to have a specific ADCC activity or increased or decreased ADCC activity as compared to a control or reference antibody composition by modulating (e.g., increasing or decreasing) the amount of glycans (e.g., galactosylated glycans, terminal β-galactose, G1, G1a, G1b and/or G2 galactosylated species, afucosylated glycans, core fucose, or a combination thereof) on the IgG1 antibody composition.

In some embodiments, the composition comprises a glycosylated and afucosylated IgG1 antibody (such as an anti-HER2 antibody, an anti-TNFα, or an anti-CD20 antibody, including trastuzumab, infliximab or rituximab) produced by the methods described herein, wherein the IgG1 antibody composition has increased or decreased ADCC activity compared to a reference IgG1 antibody composition containing antibodies having the same antibody sequence as the IgG1 antibody within the IgG1 antibody composition having increased or decreased ADCC activity.

Accordingly, in exemplary embodiments, the presently disclosed antibody compositions have increased ADCC activity to any degree or level relative to a control or a reference antibody composition. In exemplary instances, the increased ADCC activity of the antibody compositions disclosed herein (such as glycosylated and afucosylated anti-HER2, anti-TNRα, or anti-CD20 antibodies, including trastuzumab, infliximab or rituximab) using the methods of the disclosure is at least or about a 1% to about a 100% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, at least or about a 100% increase) relative to a control or a reference antibody composition. In exemplary embodiments, the increased ADCC activity of the antibody compositions disclosed herein (such as glycosylated and afucosylated anti-HER2, anti-TNFα, or anti-CD20 antibodies, including trastuzumab, infliximab or rituximab) using the methods of the disclosure is over 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody composition. In exemplary embodiments, the level of ADCC activity of the antibody compositions disclosed herein (such as glycosylated and afucosylated anti-HER2, anti-TNFα, or anti-CD20 antibodies, including trastuzumab, infliximab or rituximab) using the methods of the disclosure increases by an amount falling within the range of about 5% to about 400%, relative to a control or a reference antibody composition. In exemplary embodiments, the level of ADCC activity of the antibody composition increases by about 1.5-fold, about 2-fold, about 3-fold, about 4-fold or about 5-fold, relative to a control or a reference antibody composition. In exemplary embodiments, the level of ADCC activity of the antibody composition increases by about 6-fold, about 7-fold, about 8-fold, about 9-fold, or about 10-fold, relative to a control or a reference antibody composition. In exemplary embodiments, the level of ADCC activity of the antibody compositions disclosed herein (such as glycosylated and afucosylated anti-HER2, anti-TNFα, or anti-CD20 antibodies, including trastuzumab, infliximab or rituximab) using the methods of the disclosure increases by an amount falling within the range of about 0.5-fold to about 8-fold, relative to a control or a reference antibody composition.

In alternative embodiments, the presently disclosed antibody compositions have decreased ADCC activity to any degree or level relative to a control or a reference antibody composition. For example, the decreased ADCC activity of the antibody compositions disclosed herein (such as glycosylated and afucosylated anti-HER2, anti-TNFα, or anti-CD20 antibodies, including trastuzumab, infliximab or rituximab) using the methods of the disclosure is at least or about a 1% to about a 100% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 9.8% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, at least or about a 100% decrease) relative to the level of a control or a reference antibody composition. In exemplary embodiments, the decreased ADCC activity of the antibody compositions disclosed herein using the methods of the disclosure is over about 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to the level of a control or a reference antibody composition. In exemplary embodiments, the level of ADCC activity of the antibody compositions disclosed herein (such as glycosylated and afucosylated anti-HER2, anti-TNFα, or anti-CD20 antibodies, including trastuzumab, infliximab or rituximab) using the methods of the disclosure decreases by an amount falling within the range of about 5% to about 400%, relative to a control or a reference antibody composition. In exemplary embodiments, the level of ADCC activity of the antibody composition decreases by about 1.5-fold, about 2-fold, about 3-fold, about 4-fold or about 5-fold, relative to a control or a reference antibody composition. In exemplary embodiments, the level of ADCC activity of the antibody composition decreases by about 6-fold, about 7-fold, about 8-fold, about 9-fold, or about 10-fold, relative to a control or a reference antibody composition. In exemplary embodiments, the level of ADCC activity of the antibody compositions disclosed herein (such as glycosylated and afucosylated anti-HER2, anti-TNFα, or anti-CD20 antibodies, including trastuzumab, infliximab or rituximab) using the methods of the disclosure decreases by an amount falling within the range of about 0.5-fold to about 8-fold, relative to a control or a reference antibody composition.

In exemplary aspects, the antibody compositions of the present disclosure include antibodies having an increased amount of glycans (e.g., galactosylated glycans, G1, G1a, G1b and/or G2 galactosylated species, afucosylated glycans, core fucose, or a combination thereof) to any degree or level relative to a control or a reference antibody composition. In exemplary instances, the antibody compositions disclosed herein (such as glycosylated and afucosylated anti-HER2, anti-TNFα, or anti-CD20 antibodies, including trastuzumab, infliximab or rituximab) using the methods of the disclosure have an increased amount of glycans, wherein the glycans are increased by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or a reference antibody composition. In exemplary embodiments, the antibody compositions have an increased amount of glycans, wherein the glycans are increased by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody composition. In exemplary embodiments, the level of glycans of the antibody compositions disclosed herein (such as glycosylated and afucosylated anti-HER2, anti-TNFα, or anti-CD20 antibodies, including trastuzumab, infliximab or rituximab) using the methods of the disclosure increases by an amount falling within the range of about 5% to about 400%, relative to a control or a reference antibody composition. In exemplary embodiments, the antibody compositions have an increased amount of glycans, wherein the glycans are increased by about 1.5-fold, about 2-fold, about 3-fold, about 4-fold or about 5-fold, relative to a control or a reference antibody composition. In exemplary embodiments, the antibody compositions have an increased amount of glycans, wherein the glycans are increased by about 6-fold, about 7-fold, about 8-fold, about 9-fold, or about 10-fold, relative to a control or a reference antibody composition. In exemplary embodiments, the antibody compositions disclosed herein (such as glycosylated and afucosylated anti-HER2, anti-TNFα, or anti-CD20 antibodies, including trastuzumab, infliximab or rituximab) using the methods of the disclosure have an increased amount of glycans, wherein the glycans are increased by an amount falling within the range of about 0.5-fold to about 8-fold, relative to a control or a reference antibody composition.

In exemplary aspects, the antibody compositions of the present disclosure include antibodies having a reduced amount of glycans (e.g., galactosylated glycans, G1, G1a, G1b and/or G2 galactosylated species, afucosylated glycans, core fucose, or a combination thereof) to any degree or level relative to a control or a reference antibody composition. In exemplary instances, the antibody compositions disclosed herein (such as glycosylated and afucosylated anti-HER2, anti-TNFα, or anti-CD20 antibodies, including trastuzumab, infliximab or rituximab) have a reduced amount of glycans, wherein the glycans are reduced by at least or about 1% to about 100% (e.g., at least or about 1%, at least or about 2%, at least or about 3%, at least or about 4%, at least or about 5%, at least or about 6%, at least or about 7%, at least or about 8%, at least or about 9%, at least or about 9.5%, at least or about 9.8%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%) relative to a control or a reference antibody composition. In exemplary embodiments, the antibody compositions have a reduced amount of glycans, wherein the glycans are reduced by 100% or more, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900% or even at least or about 1000% relative to a control or a reference antibody composition. In exemplary embodiments, the glycans of the antibody compositions disclosed herein (such as glycosylated and afucosylated anti-HER2, anti-TNFα, or anti-CD20 antibodies, including trastuzumab, infliximab or rituximab) using the methods of the disclosure decreases by an amount falling within the range of about 5% to about 400%, relative to a control or a reference antibody composition. In exemplary embodiments, the antibody compositions have a reduced amount of glycans, wherein the glycans are reduced by about 1.5-fold, about 2-fold, about 3-fold, about 4-fold or about 5-fold, relative to a control or a reference antibody composition. In exemplary embodiments, the antibody compositions have a reduced amount of glycans, wherein the glycans are reduced by about 6-fold, about 7-fold, about 8-fold, about 9-fold, or about 10-fold, relative to a control or a reference antibody composition. In exemplary embodiments, the antibody compositions disclosed herein (such as glycosylated and afucosylated anti-HER2, anti-TNFα, or anti-CD20 antibodies, including trastuzumab, infliximab or rituximab) have a reduced amount of glycans falling within the range of about 0.5-fold to about 8-fold, relative to a control or a reference antibody composition.

In exemplary aspects, the antibody compositions of the present disclosure (such as glycosylated and afucosylated anti-HER2, anti-TNFα, or anti-CD20 antibodies, including trastuzumab, infliximab or rituximab) comprise a total amount of galactosylated glycans or G1, G1a, G1b and/or G2 galactosylated species of at least or about 0.5%, at least or about 1%, at least or about 2%, at least or about 3%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97% or at least or about 98% or a total amount in the range of at least or about 0.5% to 98% or a total amount in the range of 0% to 100%.

In exemplary aspects, the antibody compositions of the present disclosure (such as glycosylated and afucosylated anti-HER2, anti-TNFα, or anti-CD20 antibodies, including trastuzumab, infliximab or rituximab) comprise a total amount of galactosylated glycans or G1, G1a, G1b and/or G2 galactosylated species and afucosylated glycans, wherein the a total amount of galactosylated glycans or G1, G1a, G1b and/or G2 galactosylated species is at least or about 0.5%, at least or about 1%, at least or about 2%, at least or about 3%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97% or at least or about 98% or a total amount in the range of at least or about 0.5% to 98% or a total amount in the range of 0% to 100%; and a total amount of afucosylated glycans of at least about 5% or greater than about 5% or a total amount in the range of about 5% to 100% afucosylated glycans.

In exemplary embodiments, the antibody compositions provided herein are combined with a pharmaceutically acceptable carrier, diluent or excipient. Accordingly, provided herein are pharmaceutical compositions comprising the recombinant glycosylated protein composition (e.g., the antibody composition) described herein and a pharmaceutically acceptable carrier, diluent or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

The following examples are given merely to illustrate the present disclosure and not in any way to limit its scope.

### EXAMPLES

The following Examples describe modulating ADCC effector function of IgG1 antibodies and antibody compositions, through the increase or decrease of specific glycans, including afucosylated galactosylated glycans. The Examples demonstrate the influence of galactosylation of therapeutic IgG1 mAbs on ADCC activity by applying various glycan enrichment and remodeling tools, and then testing the impact of glycan engineered mAbs in cell-based effector function assays. Efforts were made to generate materials with desired glycan composition so that the detailed impact of terminal galactose on ADCC for both fucosylated and afucosylated mAb species could be delineated.

In the following Examples, the following materials and methods were used.

### Materials and Methods

Therapeutic monoclonal antibodies trastuzumab (anti-HER2), (rituximab (anti-CD20), and infliximab (anti-TNFα) were expressed in CHO cells and produced as high concentration solutions with standard manufacturing processes. The mAbs used in this study target receptors such as CD20, the EGFR family member HER2, and TNFα.

### Enzymatic Remodeling of Terminal Galactose Residues on Trastuzumab, Rituximab and Infliximab

Galactose remodeled series of samples for trastuzumab, rituximab and infliximab were generated by taking advantage of the in vitro degalactosylation and galactosylation capability of β-(1-4)-galactosidase (QA-Bio) and β-1,4-galactosyltransferase (Roche). To remove galactose, trastuzumab, rituximab and infliximab were first incubated with β-(1-4)-galactosidase (QA-Bio) at a ratio of 1/50 in the presence of a reaction buffer containing 50 mM sodium phosphate (pH 6.0), for 1-2 hours at 37° C. Protein A affinity chromatography purification (used to remove galactosidase and other components) was then carried out with a prepacked protein A column (Poros PrA, Applied Biosystem) on an Agilent 1100 series HPLC system with a flow rate of 3 mL/min. After injecting an appropriate amount of each sample onto the column, 100% buffer A (20 mM Tris-HCl/150 mM NaCl, pH 7.0) ran through the column for 1.4 min, followed by 100% buffer B (0.1% acetic acid) for 2.9 min, during which fractions were collected. Fractions containing eluted mAbs were diafiltered into desired buffer systems using Amicon Ultra centrifugal filters with a 3 kDa cutoff membrane.

To add galactose back at defined levels, samples were then incubated with β-1,4-galactosyltransferase (Roche) at 37° C. in a reaction buffer containing 10 mM UDP-galactose, 100 mM MES (pH 6.5), 20 mM MnCl₂ and 0.02% sodium azide. The final enzyme to mAb ratio was 6/1 (4/mg) with a mAb concentration of 2 mg/mL. MAbs with different levels of galactose were obtained by taking samples out of the reaction mixture at different time points followed by flash freezing to terminate the reaction. β-1,4-galactosyltransferase was removed by Protein A chromatography as described above. The final mAb concentration for ADCC assays are typically 1 mg/mL based on UV-Absorbance at 280 nm. Galactose remodeled samples were typically aliquoted and stored at ˜80 C before ADCC assays.

### Preparation of Afucosylated Trastuzumab and Rituximab with and without Terminal Galactose

Trastuzumab and rituximab drug substance (“DS”) were first separated into two fractions (flow-through and eluate) using a customized glycap-3A column (low density FcγIIIa receptor, 3×150 mm, Zepteon) on an Agilent 1100 series HPLC. The mobile phase A contained 20 mM Tris (pH 7.5), 150 mM NaCl, and the mobile phase B was 50 mM sodium citrate (pH 4.2). A gradient (hold at 0% B for 8 min, 0% to 18% B for 22 min) at a flow rate of 0.5 mL/min was applied to obtain both fucose-enriched (flow-through) and afucose/HM-enriched (eluate) mAbs. The eluate fraction containing both afucosylated and HM-enriched species was further enzymatically treated with Endo-H (QA-Bio, PN E-EH02) to remove high mannose species. Specifically, mAbs were incubated with Endo-H for 24 hrs at 37° C. in a reaction buffer of 50 mM sodium phosphate (pH 5.5). The final mAb concentration is 4 mg/mL.

The afucosylated mAbs with and without terminal galactose were prepared by incubating the afucosylated Endo H-treated fraction with β-(1-4)-galactosidase at different conditions. Specifically, 588 μg of afucosylated mAb1 with a volume of 60 μL was incubated with 12 μL of β-(1-4)-galactosidase (QA-Bio, 3 U/mL in 20 mM Tris-HCl, 25 mM NaCl, pH 7.5), 20 μL of 5× reaction buffer (250 mM sodium phosphate, pH 6.0) and 5 μL water at 37° C. for 2 hrs. The final mAb concentration was 6.1 mg/mL with a total volume of 97 μL. 40 μL of the reaction mixture was taken out and further purified using protein A chromatography as stated above. This material was used as afucose-enriched G1 material. For the remaining 57 μL of reaction mixture, additional fresh β-(1-4)-galactosidase enzyme (1704) and 5× reaction buffer was added followed by incubating at 37° C. for 4 hrs to ensure the complete removal of terminal galactose from afucosylated trastuzumab species. The final trastuzumab concentration of 1.2 mg/4 in the reaction mixture. The generated afucosylated G0 sample was further purified using protein A chromatography as stated above.

The control sample used for trastuzumab, containing mainly fucosylated G0F species, was also generated by incubating the flow-through fractions with β-(1-4)-galactosidase under similar conditions like afucosylated G0 sample (details can be found in the paragraph above). These type of samples, which are not expected to have ADCC activities due to the absence of HM, afucosylated and galactosylated species, were used to blend with afucose-enriched G1 and G0 at different ratios to ensure desired activity range for ADCC assay. The mAb2 G0F, G1 and G0 enriched samples were generated in a similar fashion to trastuzumab.

### Preparation of Fucosylated Trastuzumab and Rituximab with Different Levels of Terminal Galactose

Fucosylated trastuzumab and rituximab, with varying levels of galactose, were generated by collecting the flow-through fraction from a glycap-3A column and treating with β (1,4) galactosidase to remove terminal galactose. Then, G0F enriched mAbs were incubated with β-1,4-galactosyltransferase (Roche) at 37° C. in a reaction buffer containing 10 mM UDP-galactose, 100 mM MES (pH 6.5), 20 mM MnCl₂ and 0.02% sodium azide. The final enzyme to mAb ratio was 6/1 (4/mg) with a mAb concentration of 2 mg/mL. MAbs with different level of galactose were obtained by taking aliquots out of the reaction mixture at different time points followed by flash freezing to terminate the reaction. Protein A chromatography was performed, and eluates were diafiltered into desired buffer systems using Amicon Ultra centrifugal filters.

### Characterization of Enriched and Remodeled Glycan Species

All the enriched and remodeled samples were characterized with Hydrophilic Interaction Liquid Chromatography (HILIC) and Size Exclusion Chromatography (SEC) to ensure desired glycan properties and minimal level of high molecular weight species.

Glycans from mAbs were released using PNGase F (New England BioLabs) with an enzyme to substrate (E/S) ratio of 1/25 (4/μg) and labeled with 12 mg/mL 2-aminobenzoic acid (2-AA, Sigma-Aldrich) by incubating the reaction mixture at 80° C. for 75 min. 2-AA labeled glycans were separated with BEH glycan column (1.7 μm, 2.1×100 mm, Waters) on an Waters Acuity or H-Class UPLC system equipped with a fluorescence detector. The column temperature was maintained at 55° C. The mobile phase A contained 100 mM ammonium format (pH 3.0) and the mobile phase B was 100% acetonitrile. Glycans were bound to the column in high organic solvent and then eluted with an increasing gradient of aqueous ammonium formate buffer (76% B was held for 5 min, followed by a gradient from 76% to 65.5% B over 14 min).

Analysis of high molecular weight species were performed using a size exclusion column (SEC) TSK-Gel G3000SWLXL (7.8×300 mm, Tosoh Bioscience) on an Agilent 1100 HPLC system with a flow rate of 0.5 mL/min. 20-40 μg of sample was typically loaded and separated isocratically with a mobile phase containing 100 mM sodium phosphate (pH 6.8) and 250 mM NaCl.

### Experimentally Measurement of the Total Afucosylated Trastuzumab (G0 & G1) Impact on Trastuzumab's ADCC Activity

To confirm that the calculated overall impact of afucosylated trastuzumab on ADCC, which is based on the individual impact from G0 and G1 and their relative ratio for a DS lot, an experiment was designed to measure the overall afucose impact directly.

A trastuzumab DS lot, which has a afucosylated G1 and G0 at a ratio of 4:3, was treated with Endo-H (QA-Bio) followed by affinity chromatography using customized glycap-3A column (low density FcγIIIa receptor, 3×150 mm, Zepteon) on an Agilent 1100 series HPLC. The details for Endo-H treatment and FcγIIIa affinity chromatography procedures were essentially same as those described above. The afucosylated trastuzumab, including both galactosylated and non-galactosylated species without further separation, was blended with the G0F enriched mAb1 at different ratios followed by ADCC assays to measure the overall impact of both species on ADCC activities.

### In-Vitro ADCC Assays

ADCC assays were performed using FcγIIIa (158V)-expressing NK92 (M1) cells as effector cells and HCC2218 cells for trastuzumab, WIL2-S cells for rituximab, and CHO MT-3 cells for infliximab as target cells. Target cells were first labeled with calcein-AM prior to incubating with increasing concentrations from 3.3 to 2000 ng/mL for trastuzumab, from 0.0155 to 100 ng/mL for rituximab, and from 0.01024 ng/mL to 100000 ng/mL for infliximab. Effector cells were then added to opsonized target cells at an E:T ratio of 25:1 for approximately 1-2 hours. Calcein released from lysed target cells was determined by measuring the fluorescence of the reaction supernatant in an Envision (Perkin Elmer) fluorescence plate reader. Data were fitted to the mean fluorescence values using a constrained 4 parameter fit using SoftMaxPro software and reported as percentage ADCC activity relative to a reference standard as calculated by the EC50 standard/EC50 sample ratio. Each assay was performed in triplicate with the mean and standard deviation reported.

### Antigen Binding Assays

The CD20 antigen binding assay for rituximab was performed with WIL2-S cells, a human β-lymphoblastoid cell line, utilizing a competitive assay format reporting fluorescence inhibition. The test sample competes with a fixed concentration of an Alexa-488 labeled form of the reference standard for binding to the cell surface expressed CD20 on WIL2-S cells. Dose response curves were generated for the reference standard, assay control and test samples by serially diluting over 8 concentrations in PBS containing 0.5 mg/mL BSA to a final concentration range of 4.92-3000 ng/mL. The Alexa-488 labeled competitor is diluted to final in-well concentration of 100 ng/mL. Sample, competitor and WIL2-S cells diluted to 30,000 cells/well are added to a 96 well plate in duplicate and sealed with a plate sealer. The plates are then incubated for 4.5-6 hours at room temperature prior to measuring the fluorescence signal with an Acumen® eX3 imaging cytometer (TTP Labtech). A dose dependent decrease in fluorescence signal is detected with the increasing concentration of test sample. The relative CD20 binding of the test sample is reported relative to a reference standard sample. Data were fitted to the mean emission values using a 4 parameter curve fit using SoftMaxPro and reported as percent relative binding activity as calculated by IC50 standard/IC50 sample. Each sample is tested in 3 independent assays, and the final result is reported as the mean of the 3 determinations. Trastuzumab binding assays were performed in a similar fashion with the exception that the SKBR-3 are used as target cells and the dose response curve ranged from (0.016-10 μg/mL).

### Example 1

### Use of Combinations of Afucosylated-Galactosylated and Afucosylated-Agalactosylated Glycan Groups for High Precision Control of ADCC Function in IgG Molecules

While the role of glycan afucosylation in drastically enhancing ADCC activity of antibodies has been known for almost the two past decades (Mizushima et al., 2011), contributions of other components of glycan structures such as galactosylation or high mannose, was not well understood. For example, published data on ADCC impact of galactosylation were controversial. The overall complexity and heterogeneity of glycan structures is significant, with a typical monoclonal antibody often exhibiting more than 20 individual glycan species. Due to a limited understanding of glycan-ADCC relationships and overall complexity of glycan composition, accurate control of ADCC function for therapeutic antibodies remained challenging. An example of glycan structures in different glycan groups is shown in FIG. 2.

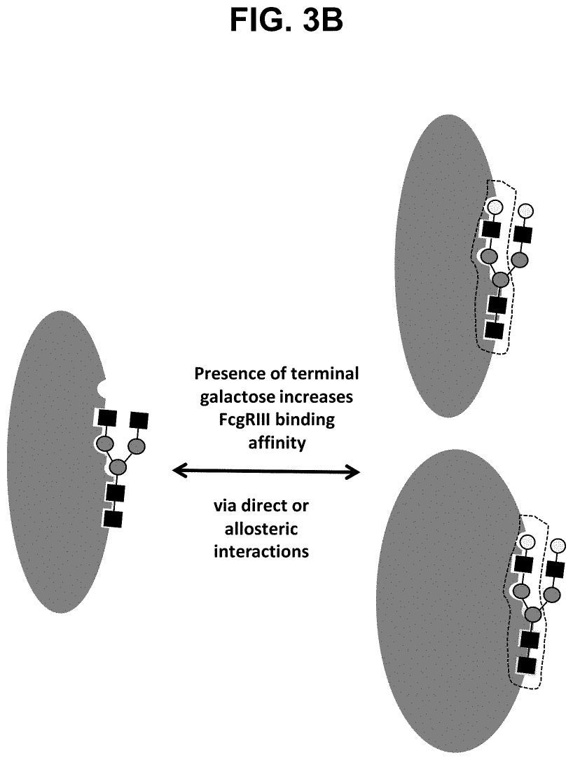

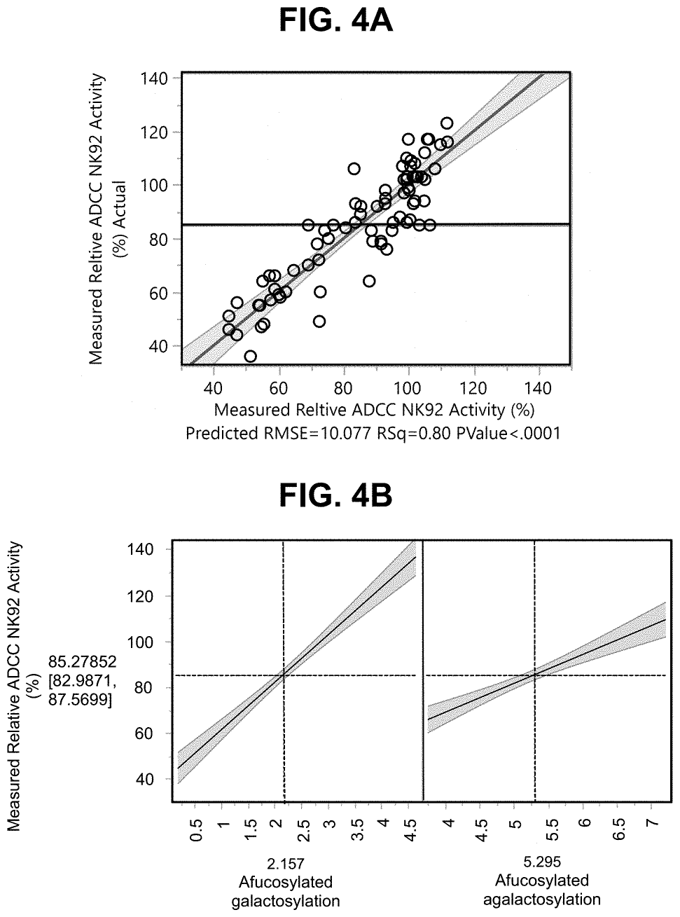

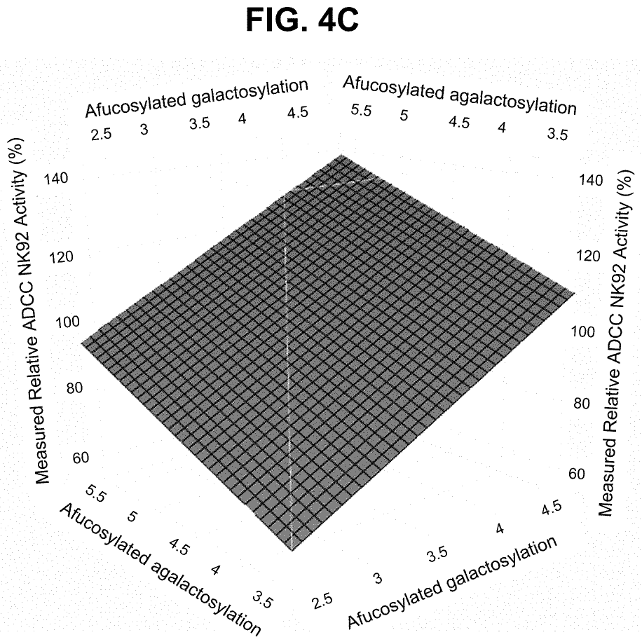

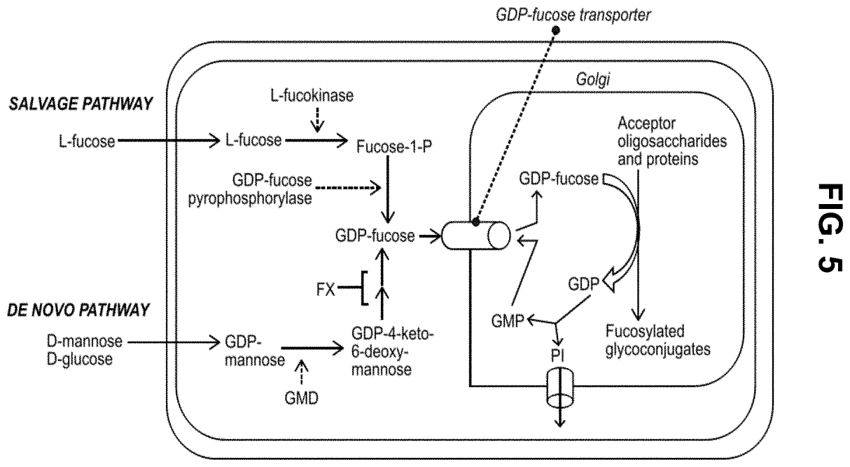

A hypothesis was formulated that galactosylation of afucosylated glycan species enhances IgG Fc region binding to the FcγRIIIa receptor (FIG. 3B) compared to agalactosylated acufosylated glycan species either via direct or allosteric interactions. Next, a modeling approach was used to screen and identify glycan species with the greatest impact on ADCC activity with an existing data set consisting of ADCC and glycan testing results for trastuzumab antibodies. Glycan-ADCC modeling confirmed that galactosylation of afucosylated species enhances ADCC by approximately 2-fold as shown in FIG. 4B.

Modeling results were confirmed by experimental glycoengineering studies with trastuzumab material. The glycoengineering studies demonstrated that the ADCC activity of purified afucosylated species with galactosylation (A2G1) is approximately 2-fold higher compared to afucosylated species without galactosylation (A2G0) based on comparison of the slopes: 20 and 13 respectively (see Examples 2-4). Additional glycoengineering studies confirmed that galactosylation of fucosylated species does not have significant impact on ADCC (data not shown).

The established model was used to define a design space for trastuzumab glycan species based on a desired target ADCC range (FIG. 4C). Using the enhanced glycan-ADCC understanding and model design space, a control strategy was developed with combined limits for afucosylation, afucosylated galactosylation and high mannose that ensured more robust control over ADCC activity compared to control of total afucosylation and high mannose.

These experiments showed that afucosylated galactosylation is a key glycan group in IgG molecules responsible for modulation of ADCC discovered via hypothesis-driven computational analysis of ADCC activity dependence on composition of individual glycan groups. It was found that afucosylated galactosylation had the strongest impact on ADCC, followed by afucosylated agalactosylation (see FIG. 2 for glycan groups definitions). Galactosylation of fucosylated glycans had minimal contribution to ADCC activity. High mannose groups had moderate-to-low contribution to ADCC and it was not practically significant if variation in the high mannose group level is <5%. This discovery was later confirmed by targeted glycoengineering experiments (as described in Examples 2-4). The afucosylated galactosylation group had approximately 2 times greater leverage of ADCC compared to afucosylated agalactosylation glycan species. Not wishing to be bound to any particular theory, the observed difference was likely driven by ability of afucosylated galactosylation to either cause a conformational change of Fc regions for a higher affinity binding to the FcγRIIIa or direct higher affinity interaction of Fc glycans with FcγRIIIa receptor. This structural hypothesis was further supported by a similar trend observed in FcγRIIIa binding assay. This knowledge is applicable to the optimal selection of glycan composition for therapeutical proteins to achieve desired functional ADCC targets for both innovator and biosimilar molecules, as well as for ADCC control strategy.

### Example 2

### The Impact of Terminal Galactose on ADCC Activity

An effective approach for studying the effect of terminal galactose on FcγIIIa receptor binding and subsequent ADCC activity has been to generate de-galacosylated and/or fully-galactosylated proteins and then to examine the galactose impact on ADCC activity. Most therapeutic IgG1s, however, contain heterogeneous populations of glycan species with no, partial- and fully-galactosylated species all present within the drug product. Furthermore, fully-galactosylated species (such as G2F) are typically only present in a small percentage compared with the other two types of species. See, e.g., Raju, T. S., et al., Glycoengineering of therapeutic glycoproteins: in vitro galactosylation and sialylation of glycoproteins with terminal N-acetylglucosamine and galactose residues. Biochemistry, 2001. 40(30): p. 8868-76. Therefore, an opportunity exists to decipher the role of all relevant glycan forms to assess the impact galactosylation on ADCC activities for IgG1s. The common forms of Fc N-linked glycan species, including those with zero, one or two galactose residues, are shown in FIG. 2.

To generate samples with different levels of galactosylation, we first removed the terminal galactose by treating a single lot of trastuzumab, rituximab and infliximab drug substance (“DS”) with galactosidase. Then, terminal galactose was attached in a controlled and selective manner using β (1, 4) galactosyltransferase (GalTase). Detailed procedures for sample preparation as well as glycan analysis are described in the Materials and Methods section of the Examples, above. Samples collected at different time-points in the Gal attachment experiments not only exhibited different levels of galactose but also contained all relevant galactosylated species, such as G1F and G2F. The levels of other critical glycan species, such as afucosylated and high mannose species on each mAb, were unaffected in these manipulations and held constant from the original DS for these samples. The galactosylation levels intentionally varied and spanned a wide range from a few percent to about 90%. Terminal sialyation was not shown due to its low level in all three mAbs.

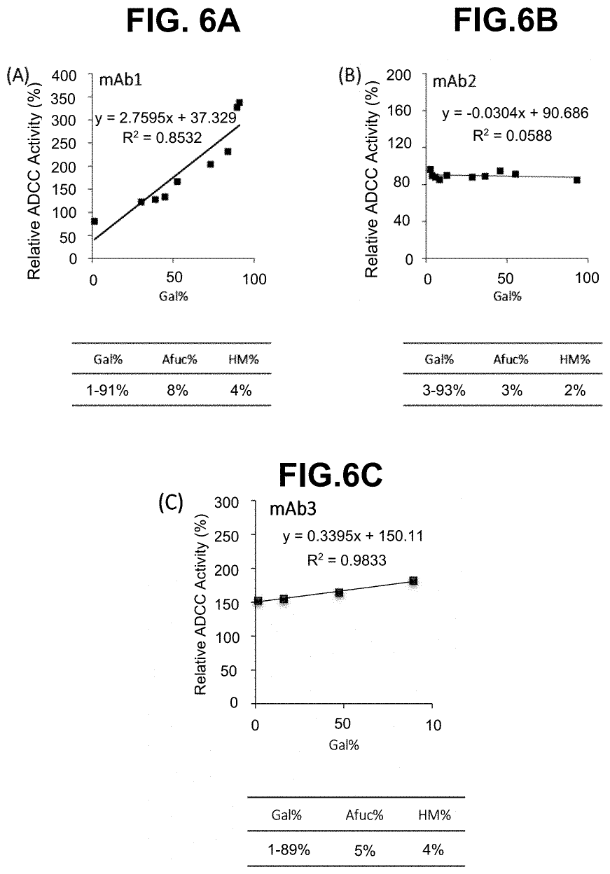

These GalTase time-course samples, with different levels of galactose trastuzumab, rituximab and infliximab, were tested by using either hydrophilic interaction chromatography (HILIC) or mass spec based glycan analysis and size exclusion chromatography (SEC) method to ensure that the desired glycan composition was achieved and no elevated high molecular weight (HMW) species were present. All the samples tested in corresponding ADCC functional assays were found to contain expected glycan species and to contain unmeasurable or minimal HMW species. Levels of glycan species including terminal galactose, afucose and high mannose, for trastuzumab (“mAb1”), rituximab (“mAb2”), and infliximab (“mAb3”) were summarized in the tables in FIGS. 6A, 6B and 6C, respectively.

Galactose re-engineered mAbs were then tested in cell-based functional assays to determine the impact of galactosylation on ADCC activity. FIG. 6 illustrates the relative ADCC activities of trastuzumab (“mAb1”), rituximab (“mAb2”), and infliximab (“mAb3”) samples, as a function of the percentage of terminal galactose (Gal % contributed from both G1F and G1 was normalized to fully galactosylated species such as G2F; see the Methods section above for Gal % calculations). A positive correlation between ADCC and Gal % was observed for both trastuzumab and infliximab. The impact coefficients (ADCC %/Gal %), represented by the slopes, were 2.8 for trastuzumab and 0.3 for infliximab, as shown in FIGS. 6A and 6C, respectively. In contrast, terminal galactose seemed to have no impact on ADCC activity for rituximab as shown in FIG. 6B. Notably, the trastuzumab and infliximab antibodies had 5% or more afucosylation, while the rituximab antibody had only 3% afucosylation (see FIGS. 6A-C).

These results indicated that the impact of terminal galactose on ADCC activity might be mAb specific. In other words, terminal galactosylation could have either a positive or no influence for a specific IgG1. However, this conclusion could only be drawn with the assumption that the terminal galactose on fucosylated and afucosylated mAbs have similar influence on their ADCC activities because both types of species were present in tested samples. In order to have a deeper understanding of terminal galactose's potential function on ADCC activities and to rule out the potential impact of fucosylation/afucosylation of ADCC activity, further studies were conducted to investigate galactosylation impact on afucosylated and fucosylated mAbs separately. See Example 3.

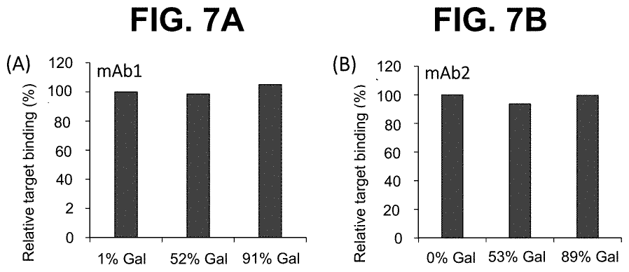

Another general concern for glycan reengineering/manipulation is the potential risk to inadvertently or indirectly affecting antigen binding which may lead to reduced target cell binding and effector function activity. Competitive antigen binding assays for both trastuzumab and rituximab were performed to test relative binding activities of terminal galactose reengineered mAbs to their corresponding antigens. No appreciable changes were observed for representative samples with low, medium and high levels of galactosylation for both trastuzumab and rituximab as shown in FIGS. 7A and 7B, respectively.

### Example 3

### The Impact of Terminal Galactose on ADCC Activity for Afucosylated mAbs

To assess the influence of galactose on afucosylated mAb species, both afucosylated species with and without terminal galactose were generated. Two factors were considered in the experimental design. First, we focused on the impact of relevant glycan species by using lots from a representative mAb production process as starting materials, in which the afucosylated G0 and G1 glycans were the dominant afucosylated species. Both afucosylated trastuzumab without galactose (G0) and with galactose (G1) were enriched, while other glycan attributes (such as high mannose) were kept consistent. Second, enriched afucosylated G0 and G1 mAbs could not be measured by the ADCC assay directly because the ADCC response would be out of the assay working range due to the high content of afucosylated species (which are known to have significant impact on ADCC activity). Therefore, mAbs enriched with G0F glycans, which is expected to have minimal ADCC activity, was then blended with samples enriched with afucosylated G0 and G1 species at different ratios to allow ADCC activity of blended materials to fall into the working range for each mAb's ADCC assay.

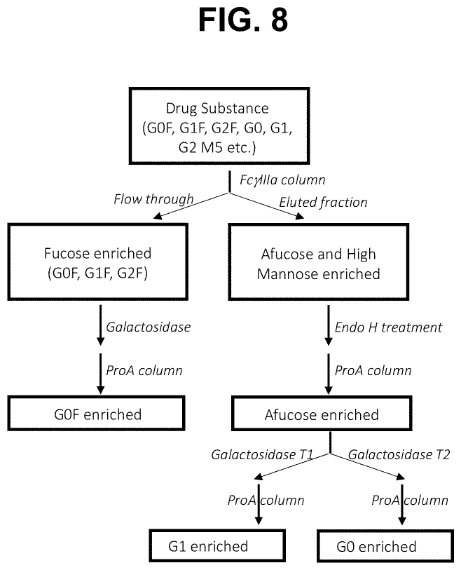

The enrichment procedures of afucosylated and fucosylated mAbs are illustrated in the workflow in FIG. 8. First, FcγIIIa receptor-based affinity chromatography was used to separate fucosylated species from afucosylated and high mannose species. See, e.g., Bolton, G. R., et al., Separation of nonfucosylated antibodies with immobilized FcgammaRIII receptors. Biotechnol Prog, 2013. 29(3): p. 825-828. Galactose in the fucosylated fraction was then removed using galactosidase to generate mAbs with G0F as the dominant glycoform. Next, afucosylated species were further enriched by first removing high mannose glycans from the mAbs with endo-H treatment in the eluted fraction from the FcγIIIa receptor column. Endo H-treated samples were then subsequently treated with galactosidase under slightly different enzymatic conditions (experimental details were described in Methods section) to generate afucosylated G0 and G1 enriched mAb samples, which were then blended with mAbs enriched with G0F at three different ratios before measuring ADCC activity. Intact mass analysis on mAbs was conducted to closely monitor each step and the enriched materials were further characterized by HILIC/mass spec-based glycan analysis.

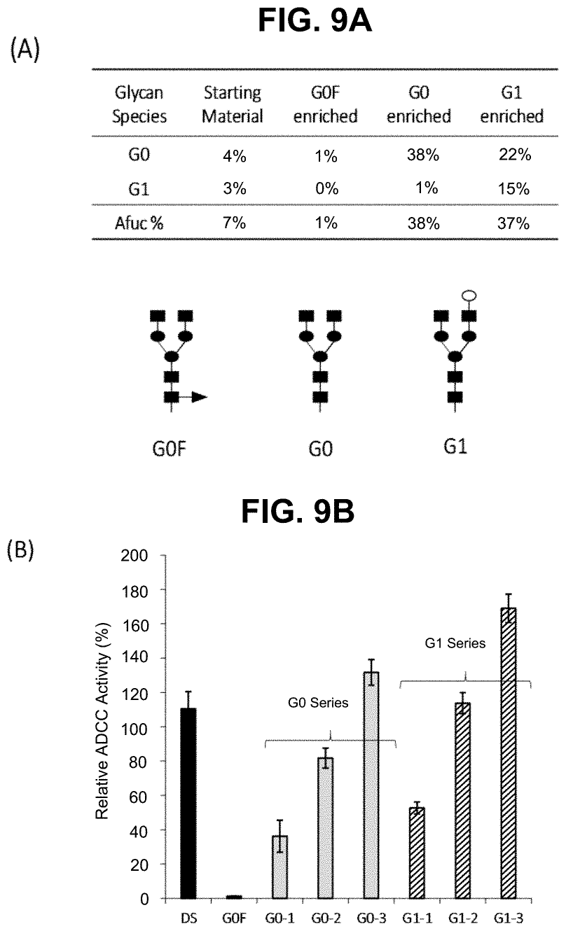

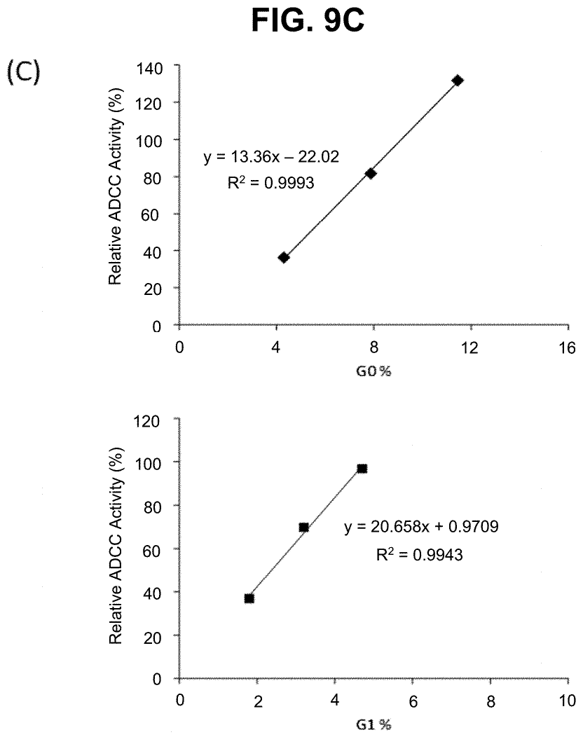

To determine the impact of terminal galactose on ADCC activity for trastuzumab, G0F, G0 and G1 enriched samples were generated as described in the Methods section above. Relevant glycan information is shown in FIG. 9A. The total afucosylation levels were comparable for G0 and G1 enriched samples (37-38%), while the level of the G1 glycoform was 15% for the G1 enriched sample and 1% for the G0 enriched sample. The G0 enriched sample was blended with the G0F enriched sample at three different ratios to generate a G0 series of samples, which contained 5% afucosylated trastuzumab for G0-1, 11% for G0-2 and 16% for G0-3. Similarly, a G1 series of samples were generated by blending the G1 enriched sample with G0F, and the afucosylation level was 5%, 11% and 16% for G1-1, G1-2 and G1-3, respectively.

The ADCC activities of G0 series of samples (G0-1, G0-2, G0-3) and G1 series of samples (G1-1, G1-2, G1-3), were analyzed side by side. Relative to the reference standard (typically a well characterized DS lot that serves as the primary reference for other DS lots and DP lots) for trastuzumab, the starting material of trastuzumab DS lot showed ADCC activity of 111% on average, whereas the G0F enriched sample had an average activity of 1% (FIG. 9B, black bars). This is consistent with our expectation that initial DS material has similar activity as a trastuzumab reference standard and that G0F enriched mAbs have minimal ADCC activity due to the removal of trastuzumab species with high mannose, afucosylated and galactosylated glycan structures. Moreover, G1 enriched trastuzumab samples, including G1-1, G1-2 and G1-3, consistently showed relative higher ADCC activity (FIG. 9B, pattern bars) than the corresponding G0 samples G0-1, G0-2 and G0-3 (FIG. 9B, grey bars). In addition, a quantitative linear correlation was observed for ADCC activity with G0 content (top panel) and with G1 content (bottom panel) as illustrated in FIG. 9C. The impact coefficient, defined as % ADCC/% glycan, is about 13 for G0 (FIG. 9C top, slope) and about 21 for G1 (FIG. 9C bottom, slope). Together, results obtained here indicate that afucosylated glycan with terminal galactose has higher impact than that without terminal galactose. The ratio of G1:G0 activity coefficients is approximately 1.6 for trastuzumab.

These results showed that the afucosylated G0 and G1 glycan species are both capable of stimulating ADCC activity and that addition of galactose to the afucosylated complex glycan is more active. Therefore, the overall impact of afucosylation for a specific mAb on its ADCC activity would be influenced by the relative levels of afucosylated G1 and G0 species as both glycoforms were typically present in its DS lot. For the DS lot used in this study, the relative ratio for G0 and G1 species is 4:3 for trastuzumab. Therefore, based on the individual impact slopes in FIG. 9C and the relative ratio of G1 vs G0, the overall impact coefficients (ADCC %/Afuc %) for afucosylation on trastuzumab's ADCC activity can be calculated: 13*4/7+21*3/7=16.

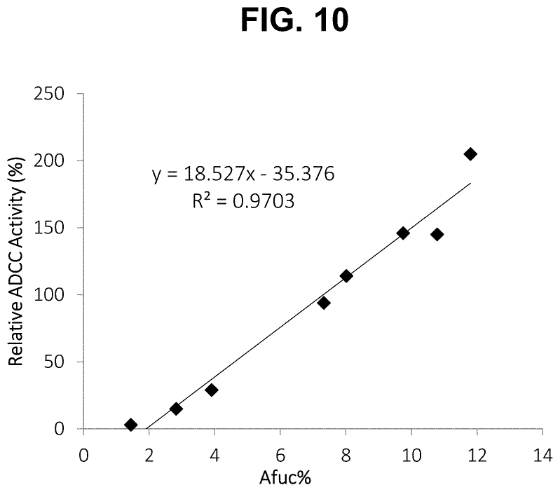

This impact coefficient for afucosylation on trastuzumab ADCC was also measured experimentally. Briefly, afucosylated trastuzumab, including both G0 and G1 forms, was enriched and then blended with G0F enriched trastuzumab to generate a series of samples with different level of afucosylation, which were then analyzed using its ADCC assay. The result, as shown in FIG. 10, indicated that the measured impact coefficients for afucosylation is 18, which agreed with the calculated impact factor overall. The experimental details were described in the Methods section, above.

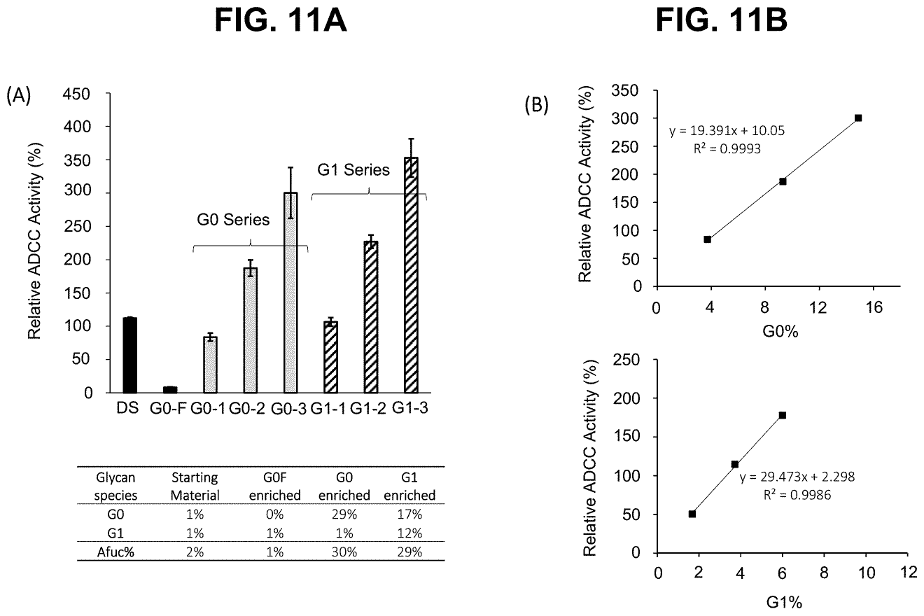

Similar to trastuzumab, the afucose and galactose remodeling experiment (as outlined in FIG. 8) was also performed on rituximab to understand the impact of galactosylation associated with afucosylated rituximab. The afucosylated glycan levels for these enriched materials are shown in FIG. 11A (bottom). The G1 enriched rituximab material had 12% G1 out of total 29% afucosylated species, while G0 enriched material had only 1% G1 and 29% G0. This indicated a successful enrichment of desired species even when the starting material contained a low level (2%) of afucosylated species. Similar to what was done for trastuzumab, G0 and G1 enriched samples were then blended at three different ratios with G0F. These blended samples contained final afucosylation levels of 5% for G0-1 and G1-1, 10% for G0-2 and G1-2, and 15% for G0-3 and G1-3. An ADCC assay, specific for rituximab, was used to measure ADCC activities for the G0 series of samples (G0-1, G0-2, G0-3), the G1 series of samples (G1-1, G1-2, G1-3), the starting DS material, and the G0F enriched material.

Like trastuzumab, the G1 series for rituximab showed overall higher ADCC activities than the corresponding G0 series (FIG. 11A). The activity coefficients (FIG. 11B, slopes) between rituximab ADCC activity and glycan level were 19 for G0 (top) and 29 for G1 (bottom). The impact ratio for G1 versus G0 was 1.5 for rituximab, which is similar to that for trastuzumab (1.6). Taken together, the results for trastuzumab and rituximab showed that terminal galactose had a meaningful impact on ADCC activities for afucosylated mAbs.

### Example 4

### The Impact of Terminal Galactose on ADCC Activity for Fucosylated mAbs

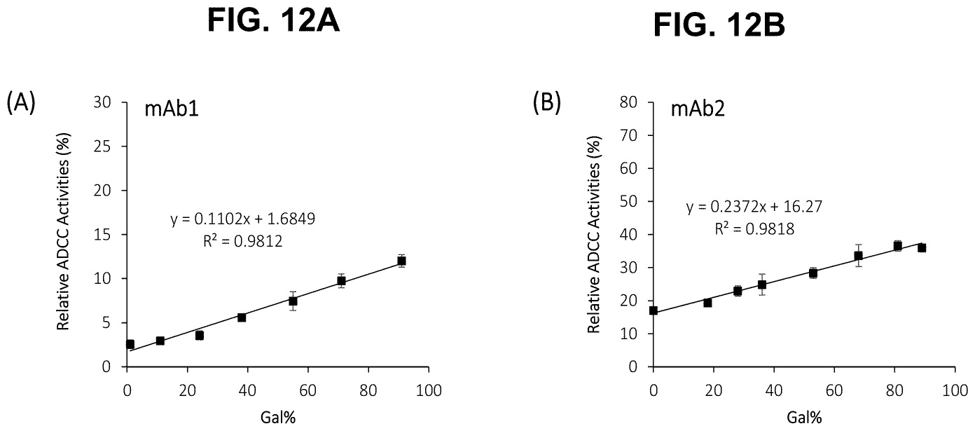

Assessment of terminal galactose impact on ADCC activities for fucosylated trastuzumab and rituximab was also performed. First, G0F enriched trastuzumab and rituximab was generated from their corresponding DS lots by collecting the flow-through from FcγIIIa affinity chromatography followed by galactosidase treatment and cleaning up with ProA chromatography (as illustrated in FIG. 8, left). Next, terminal galactose was added to G0F species through enzymatic remodeling of mAbs with β (1, 4) galactosyltransferase. Samples with different levels of terminal galactose were achieved by controlling the incubation time for such enzymatic reactions. Finally, the impact of terminal galactose on fucosylated trastuzumab and rituximab was evaluated by measuring the ADCC activities of these samples with different Gal % using their corresponding functional assays. The results obtained for trastuzumab (“mAb1”) and rituximab (“mAb2”) are shown in FIGS. 12A and 12B, respectively. The relative ADCC activities for these fucosylated samples compared with reference standards were low for both trastuzumab and rituximab. The maximum ADCC activity with the highest level of terminal galactose (˜90%) is less than 15% for trastuzumab and less than 40% for rituximab. Moreover, unlike the terminal Gal impact on afucosylated trastuzumab and rituximab (FIGS. 9 and 11), where impact coefficients (ADCC %/Gal %) were more than 20, the terminal Gal impacts on fucosylated trastuzumab and rituximab were minimal with impact coefficients of about 0.1 for mAb1 and 0.2 for mAb2 (FIGS. 12A and 12B). This result indicated that galactosylation with a range of 0-90% is unlikely to have any meaningful impact on ADCC activities when it is associated with fucosylated mAbs. In other words, the impact of terminal galactose on ADCC activity was significantly influenced by the absence of core fucose and its impact on fucosylated mAbs was negligible.

The following is a discussion of the results of Examples 2-4.

Thorough understanding of structure-function relationships of product quality attributes is of critical importance for protein therapeutic development under the quality by design (QbD) paradigm. While N-linked glycosylation in the Fc region of IgGs is well known to play an important role in modulating antibody effector functions such as ADCC, contributions of individual glycan species have not been thoroughly understood. This is, due in part, the complexity and heterogeneity of the glycan structures typically observed with therapeutic antibodies. A comprehensive understanding of the impact of individual glycan species on biological functions could help to optimize control strategies by focusing on the most relevant attributes to achieve a desired range of effector function.

In this study, interrelated effects between galactosylation and afucosylation on ADCC were revealed for both trastuzumab and rituximab. Terminal galactose increased ADCC activity mainly when it is present on afucosylated mAbs but not on fucosylated species. The identification of galactosylation associated with afucosylated IgG1s as a critical quality attribute is a significant advancement towards understanding how Fc glycans mediate ADCC. Such an in-depth understanding will help facilitate establishment of attribute-focused development strategies for biotherapeutics and ensures that the target product profile is achieved by controlling important attributes such as afucosylated terminal galactosylation. Meanwhile, it also allows appropriate flexibility or design space for non-critical glycan attributes such as galactosylation associated with fucosylated species. These results highlight the need to distinguish terminal galactose on afucosylated species from that on fucosylated species when the impact of terminal galactose on ADCC activities is to be assessed for mAbs. In addition, once the impact coefficients of afucosylated glycans with and without terminal galactose on a specific mAb's ADCC are obtained, they could be used to calculate the overall impact coefficients of afucosylation. This has been as confirmed by using trastuzumab for example, where the calculated impact coefficient of afucosylation has a good agreement with the experimentally measured value. This is especially useful when the ratio of afucosylated G0 to G1 varies from lot to lot, because the over impact of afucosylation can then be predicted without the need to conduct time-consuming experiments.

Interrelated glycan effects, observed for both trastuzumab and rituximab, may be applicable to other IgG1 molecules given that trastuzumab and rituximab target different antigens and have different molecule specific ADCC assays with different target cells for evaluating the impact of terminal galactosylation.

It is also worth noting that identification of afucosylated terminal galactose as a critical glycan attribute for ADCC activities is consistent with the results from overall terminal galactose impacts assessment for trastuzumab (“mAb1”), rituximab (“mAb2”), and infliximab (“mAb3”) as shown in FIG. 6, where the afucosylated and fucosylated terminal galactose were not separated. Specifically, the degree of impact of terminal galactose on ADCC activities was in the order of trastuzumab (“mAb1”)>infliximab (“mAb3”)>rituximab (“mAb2”) as indicated by their corresponding slopes, which follows the same trend as the percentage of afucosylated species in trastuzumab (“mAb1”) (8%), infliximab (“mAb3”) (5%) and rituximab (“mAb2’) (3%). The contribution from fucosylated galactosylation was negligible. It is notable that the overall galactose impact coefficients on rituximab ADCC was low (˜0) as shown in FIG. 6B. Without being bound to a theory, this observation could be due to the lower content of afucosylated glycoforms in this sample compounded with molecule specific ADCC assay variations and background noises. These findings further highlight that any efforts to study terminal galactose impact on ADCC function will need to account for the core fucose. The findings obtained in this study could also explain why the influence of terminal galactose on ADCC activity has been inconsistently reported (either no impact or positive impact). As β (1, 4) galactosyltransferase treatment is able to add terminal galactose to both fucosylated and afucosylated species (Warnock, D., et al., In vitro galactosylation of human IgG at 1 kg scale using recombinant galactosyltransferase. Biotechnol Bioeng, 2005. 92(7): p. 831-42), the overall galactosylation influence on a specific mAb's ADCC will depend on the relative level of afucosylated and fucosylated glycan species: when a mAb has minimal level of afucosylated glycan species, the galactose impact detected could be negligible; when a mAb has higher levels of afucosylated species (e.g., 5% afucosylation or more), a significant impact could be expected.

The structural bases for how galactose may exert this influence on ADCC activities of afucosylated and fucosylated mAbs are not obvious. Terminal galactose on IgG1s could directly bind the FcγIIIa receptor, and/or could indirectly affect the binding of IgG1s to the receptor by causing conformational changes in the antibody. Based on the crystal structures of IgG1 Fc fragments with several different glycan forms, it was suggested that galactosylation (mainly fucosylated) could lead to increased spatial distance between CH2 domains in the Fc construct which may expose more amino acid residues to bind to the FcγIIIa receptor. See, e.g., Krapp, S., et al, Structural analysis of human IgG-Fc glycoforms reveals a correlation between glycosylation and structural integrity. 2003, 325, 979-989. On the other hand, no conformational differences were observed for an afucosylated IgG with different levels of terminal galactose (G0, G1 and G2) by using hydrogen/deuterium exchange (H/DX). See, e.g., Houde, D., et al., Post-translational modifications differentially affect IgG1 conformation and receptor binding. Mol Cell Proteomics, 2010. 9(8): p. 1716-28. The discovery of the unique carbohydrate-carbohydrate interaction between the receptor and the mAb provided a clear picture of why up to 100-fold gain in binding affinity of afucosylated vs. fucosylated IgG1s to Fcγ receptors. Ferrara, C., et al, Unique carbohydrate-carbohydrate interactions are required for high affinity binding between FcγRIII and antibodies lacking core fucose. PNAS, 2011 (108), p. 1 2669-12674.

Glycosylation is one of the major post-translational modifications and has significant potential effects on protein folding, conformation, distribution, stability and activity. Given that IgG1 with both G0 and G1 glycan forms exist at least at low levels in human serum (see, e.g., Flynn, G. C., et al, Naturally occurring glycan forms of human immunoglobulins G1 and G2. 2010, Molecular Immunology, 2010 (47), 2074-2082), it may be informative to consider the relevance of the interrelated glycan impact for galactosylation and afucosylation on naturally occurring antibodies' effector functions. Such an interplay might allow the immune system to have a finer grade of regulation and control over such kind of critical cellular activities. Future experiments using additional antibodies and relevant experimental systems need to be conducted to reveal whether such a degree of control is present in adaptive immune responses involving ADCC.

In summary, a comprehensive assessment of the biological impact of terminal galactose on ADCC activities was conducted in multiple IgG1s. Our results indicate that the degree of influence of terminal galactose on ADCC activity depends on the absence or presence of a core-fucose structure. Terminal galactose on afucosylated mAbs showed significant impact on ADCC activity, while minimal impact was observed for terminal galactose associated with fucosylated glycan structures. Such in-depth knowledge of how glycan structures influence biological activity plays a key role in the establishment of target product quality profiles in QbD paradigm and to ensuring attribute-focused product development.

### SELECTIVE REFERENCES

The following references are cited throughout the background and examples.
1. Jefferis, R., Glycosylation as a strategy to improve antibody-based therapeutics. Nat Rev Drug Discov, 2009. 8(3): p. 226-34.
2. Natsume, A., R. Niwa, and M. Satoh, Improving effector functions of antibodies for cancer treatment: Enhancing ADCC and CDC. Drug Des Devel Ther, 2009. 3: p. 7-16.
3. Flynn, G. C., et al., Naturally occurring glycan forms of human immunoglobulins G1 and G2. Mol Immunol, 2010. 47(11-12): p. 2074-82.
4. Read, E. K., J. T. Park, and K. A. Brorson, Industry and regulatory experience of the glycosylation of monoclonal antibodies. Biotechnol Appl Biochem, 2011. 58(4): p. 213-9.
5. Dicker, M. and R. Strasser, Using glyco-engineering to produce therapeutic proteins. Expert Opin Biol Ther, 2015. 15(10): p. 1501-16.
6. Ferrara, C., et al., Unique carbohydrate-carbohydrate interactions are required for high affinity binding between FcgammaRIII and antibodies lacking core fucose. Proc Natl Acad Sci USA, 2011. 108(31): p. 12669-74.
7. Okazaki, A., et al., Fucose depletion from human IgG1 oligosaccharide enhances binding enthalpy and association rate between IgG1 and FcgammaRIIIa. J Mol Biol, 2004. 336(5): p. 1239-49.
8. Shields, R. L., et al., Lack of fucose on human IgG1 N-linked oligosaccharide improves binding to human Fcgamma RIII and antibody-dependent cellular toxicity. J Biol Chem, 2002. 277(30): p. 26733-40.
9. Shinkawa, T., et al., The absence of fucose but not the presence of galactose or bisecting N-acetylglucosamine of human IgG1 complex-type oligosaccharides shows the critical role of enhancing antibody-dependent cellular cytotoxicity. J Biol Chem, 2003. 278(5): p. 3466-73.
10. Kanda, Y., et al., Comparison of biological activity among nonfucosylated therapeutic IgG1 antibodies with three different N-linked Fc oligosaccharides: the high-mannose, hybrid, and complex types. Glycobiology, 2007. 17(1): p. 104-18.
11. Pace, D., et al., Characterizing the effect of multiple Fc glycan attributes on the effector functions and FcgammaRIIIa receptor binding activity of an IgG1 antibody. Biotechnol Prog, 2016. 32(5): p. 1181-1192.
12. Zhou, Q., et al., Development of a simple and rapid method for producing non-fucosylated oligomannose containing antibodies with increased effector function. Biotechnol Bioeng, 2008. 99(3): p. 652-65.
13. Kaneko, Y., F. Nimmerjahn, and J. V. Ravetch, Anti-inflammatory activity of immunoglobulin G resulting from Fc sialylation. Science, 2006. 313(5787): p. 670-3.
14. Scallon, B. J., et al., Higher levels of sialylated Fc glycans in immunoglobulin G molecules can adversely impact functionality. Mol Immunol, 2007. 44(7): p. 1524-34.
15. Boyd, P. N., A. C. Lines, and A. K. Patel, The effect of the removal of sialic acid, galactose and total carbohydrate on the functional activity of Campath-1H. Mol Immunol, 1995. 32(17-18): p. 1311-8.
16. Hodoniczky, J., Y. Z. Zheng, and D. C. James, Control of recombinant monoclonal antibody effector functions by Fc N-glycan remodeling in vitro. Biotechnol Prog, 2005. 21(6): p. 1644-52.
17. Raju, T. S., Terminal sugars of Fc glycans influence antibody effector functions of IgGs. Curr Opin Immunol, 2008. 20(4): p. 471-8.
18. Kumpel, B. M., et al., The biological activity of human monoclonal IgG anti-D is reduced by beta-galactosidase treatment. Hum Antibodies Hybridomas, 1995. 6(3): p. 82-8.
19. Thomann, M., et al., Fc-galactosylation modulates antibody-dependent cellular cytotoxicity of therapeutic antibodies. Mol Immunol, 2016. 73: p. 69-75.
20. Thomann, M., et al., In vitro glycoengineering of IgG1 and its effect on Fc receptor binding and ADCC activity. PLoS One, 2015. 10(8): p. e0134949.
21. Houde, D., et al., Post-translational modifications differentially affect IgG1 conformation and receptor binding. Mol Cell Proteomics, 2010. 9(8): p. 1716-28.
22. Raju, T. S., et al., Glycoengineering of therapeutic glycoproteins: in vitro galactosylation and sialylation of glycoproteins with terminal N-acetylglucosamine and galactose residues. Biochemistry, 2001. 40(30): p. 8868-76.
23. Warnock, D., et al., In vitro galactosylation of human IgG at 1 kg scale using recombinant galactosyltransferase. Biotechnol Bioeng, 2005. 92(7): p. 831-42.
24. Ferrara, C., et al, Unique carbohydrate-carbohydrate interactions are required for high affinity binding between FcγRIII and antibodies lacking core fucose. PNAS, 2011 (108), p. 1 2669-12674
25. Flynn, G. C., et al, Naturally occurring glycan forms of human immunoglobulins G1 and G2. 2010, Molecular Immunology, 2010 (47), 2074-2082
26. Bolton, G. R., et al., Separation of nonfucosylated antibodies with immobilized FcgammaRIII receptors. Biotechnol Prog, 2013. 29(3): p. 825-828
27. Krapp, S., et al, Structural analysis of human IgG-Fc glycoforms reveals a correlation between glycosylation and structural integrity. 2003, 325, 979-989

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the disclosed embodiments. The section headings used herein are for organizational purposes only and are not to be construed as limiting the subject matter described.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. (canceled)

2. (canceled)

3. A method for engineering a specific target Antibody Dependent Cellular Cytotoxicity (ADCC) activity of a glycosylated and afucosylated IgG1 antibody composition comprising: (1) determining the ADCC activity of a glycosylated and afucosylated IgG1 antibody composition; (2) determining a target ADCC activity; and (3) increasing or decreasing the ADCC activity of the glycosylated and afucosylated IgG1 antibody composition by increasing or decreasing the amount of terminal β-galactose in the glycan species at the consensus glycosylation site, wherein the ADCC activity of the glycosylated and afucosylated IgG1 antibody composition after increasing or decreasing the amount of terminal β-galactose is the same as the target ADCC activity or within about 10%, about 15%, about 20%, about 25%, about 30%, about 35%, about 40%, about 45% or about 50% of the target ADCC activity or within about 1% to about 50% of the target ADCC activity.

4. The method of claim 3, wherein step 1 occurs before, after or at the same time as step 2 and/or step 3; or step 2 occurs before, after or at the same time as step 1 and/or step 3.

5. The method accordingly to claim 3, wherein an increase of about 1% β-galactose in afucosylated glycans increases ADCC activity by about 20% to about 30%.

6. The method accordingly to claim 3, wherein a decrease of about 1% β-galactose in afucosylated glycans decreases ADCC activity by about 20% to about 30%.

7. The method accordingly to claim 3, wherein the terminal β-galactose in the glycan species is a G1, G1a, G1b, G2 or hybrid galactosylated species of the IgG1 antibody.

8. The method accordingly to claim 3, wherein the IgG1 antibody is produced in a eukaryotic host cell.

9. (canceled)

10. (canceled)

11. The method accordingly to claim 3, wherein the IgG1 antibody composition comprises an anti HER2 antibody, an anti-TNFα antibody, or an anti-CD20 antibody.

12. The method accordingly to claim 3, wherein the IgG1 antibody composition comprises trastuzumab, infliximab, or rituximab.

13. The method accordingly to claim 3, wherein the ADCC activity of the antibody composition is increased or decreased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 1-fold, about 2-fold, about 3-fold, or about 4-fold, or increased or decreased by about 5% to about 400%.

14. The method accordingly to claim 3, wherein the amount of terminal β-galactose in the antibody composition is increased or decreased by about 10%, about 15%, about 20%, about 25%, about 30%, about 35%, about 40%, about 45%, about 50%, about 55%, about 60%, about 65%, about 70%, about 75%, about 80%, about 85%, about 90% about 95%, about 100%, about 125%, about 150%, about 175% or about 200%; or increased or decreased to a total amount of about 0.5%, about 1%, about 2%, about 3%, about 5%, about 7%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97% or about 98% or increased or decreased to a total amount of about 0% to 100%.

15. The method according to claim 3, wherein the ADCC activity is measured or determined using a cell-based assay or a binding assay.

16. The method of claim 15, wherein the cell-based assay comprises NK92 or PMBC cells.

17. The method of claim 15, wherein the binding assay comprises FcγRIIIa.

18. The method accordingly to claim 3, wherein the amount of terminal β-galactose in the antibody composition is increased or decreased by culturing cells expressing an antibody in cell culture media that modulates the amount of terminal β-galactose in the glycan species of the antibody.

19. The method accordingly to claim 3, wherein the amount of terminal β-galactose is increased or decreased using a chemical or an enzyme.

20. The method of claim 18, wherein the enzyme is selected from the group consisting of: Endo-S2; β-(1-4)-Galactosidase; Endo-H; β-1,4-galactosyltransferase; and PNGase F.

21. (canceled)

22. (canceled)

23. (canceled)

24. (canceled)

25. The method of claim 12, wherein the trastuzumab antibody comprises:
a. a light chain variable domain comprising: (i) a light chain CDR1 sequence comprising the amino acid sequence set forth in SEQ ID NO:1; (ii) a light chain CDR2 sequence comprising the amino acid sequence set forth in SEQ ID NO:2; and (iii) a light chain CDR3 sequence comprising the amino acid sequence set forth in SEQ ID NO:3; and
b. a heavy chain variable domain comprising: (i) a heavy chain CDR1 sequence comprising the amino acid sequence set forth in SEQ ID NO: 4; (ii) a heavy chain CDR2 sequence comprising the amino acid sequence set forth in SEQ ID NO:5, and (iii) a heavy chain CDR3 sequence comprising the amino acid sequence set forth in SEQ ID NO:6; or
c. a light chain variable domain comprising SEQ ID NO: 7; and
d. a heavy chain variable domain comprising SEQ ID NO: 8;
or wherein the rituximab antibody comprises:
a. a light chain variable domain comprising: (i) a light chain CDR1 sequence comprising the amino acid sequence set forth in SEQ ID NO: 11; (ii) a light chain CDR2 sequence comprising the amino acid sequence set forth in SEQ ID NO: 12; and (iii) a light chain CDR3 sequence comprising the amino acid sequence set forth in SEQ ID NO: 13; and
b. a heavy chain variable domain comprising: (i) a heavy chain CDR1 sequence comprising the amino acid sequence set forth in SEQ ID NO: 14; (ii) a heavy chain CDR2 sequence comprising the amino acid sequence set forth in SEQ ID NO: 15, and (iii) a heavy chain CDR3 sequence comprising the amino acid sequence set forth in SEQ ID NO: 16; or
c. a light chain variable domain comprising SEQ ID NO: 17; and
d. a heavy chain variable domain comprising SEQ ID NO: 18;
or wherein the infliximab antibody comprises:
a. a light chain variable domain comprising: (i) a light chain CDR1 sequence comprising the amino acid sequence set forth in SEQ ID NO: 25; (ii) a light chain CDR2 sequence comprising the amino acid sequence set forth in SEQ ID NO: 26; and (iii) a light chain CDR3 sequence comprising the amino acid sequence set forth in SEQ ID NO: 27; and
b. a heavy chain variable domain comprising: (i) a heavy chain CDR1 sequence comprising the amino acid sequence set forth in SEQ ID NO: 28; (ii) a heavy chain CDR2 sequence comprising the amino acid sequence set forth in SEQ ID NO: 29, and (iii) a heavy chain CDR3 sequence comprising the amino acid sequence set forth in SEQ ID NO: 30; or
c. a light chain variable domain comprising SEQ ID NO: 31; and
d. a heavy chain variable domain comprising SEQ ID NO: 32.

26. (canceled)


SEQUENCE LISTING (34 sequences)

SEQ ID NO: 1 - protein, 6 aa, Artificial Sequence
  Synthetic Polypeptide
  MISC_FEATURE: LC CDR1
QDVNTA

SEQ ID NO: 2 - protein, 3 aa, Artificial Sequence
  Synthetic Polypeptide
  MISC_FEATURE: LC CDR2
SAS

SEQ ID NO: 3 - protein, 9 aa, Artificial Sequence
  Synthetic Polypeptide
  MISC_FEATURE: LC CDR3
QQHYTTPPT

SEQ ID NO: 4 - protein, 8 aa, Artificial Sequence
  Synthetic Polypeptide
  MISC_FEATURE: HC CDR1
GFNIKDTY

SEQ ID NO: 5 - protein, 8 aa, Artificial Sequence
  Synthetic Polypeptide
  MISC_FEATURE: HC CDR2
IYPTNGYT

SEQ ID NO: 6 - protein, 13 aa, Artificial Sequence
  Synthetic Polypeptide
  MISC_FEATURE: HC CDR3
SRWGGDGFYAMDY

SEQ ID NO: 7 - protein, 107 aa, Artificial Sequence [sequence omitted]

SEQ ID NO: 8 - protein, 120 aa, Artificial Sequence [sequence omitted]

SEQ ID NO: 9-10 - skipped (2 entries)

SEQ ID NO: 11 - protein, 10 aa, Artificial Sequence
  Synthetic Polypeptide
  MISC_FEATURE: LC-CDR1
RASSSVSYIH

SEQ ID NO: 12 - protein, 7 aa, Artificial Sequence
  Synthetic Polypeptide
  MISC_FEATURE: LC CDR2
ATSNLAS

SEQ ID NO: 13 - protein, 9 aa, Artificial Sequence
  Synthetic Polypeptide
  MISC_FEATURE: LC CDR3
QQWTSNPPT

SEQ ID NO: 14 - protein, 5 aa, Artificial Sequence
  Synthetic Polypeptide
  MISC_FEATURE: HC CDR1
SYNMH

SEQ ID NO: 15 - protein, 17 aa, Artificial Sequence
  Synthetic Polypeptide
  MISC_FEATURE: HC CDR2
AIYPGNGDTSYNQKFKG

SEQ ID NO: 16 - protein, 12 aa, Artificial Sequence
  Synthetic Polypeptide
  MISC_FEATURE: HC CDR3
STYYGGDWYFNV

SEQ ID NO: 17 - protein, 106 aa, Artificial Sequence [sequence omitted]

SEQ ID NO: 18 - protein, 121 aa, Artificial Sequence [sequence omitted]

SEQ ID NO: 19-20 - skipped (2 entries)

SEQ ID NO: 21 - protein, 214 aa, Artificial Sequence
  Synthetic Polypeptide
  MISC_FEATURE: VL-IgG Kappa
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 450 aa, Artificial Sequence
  Synthetic Polypeptide
  MISC_FEATURE: VH-IgG1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 213 aa, Artificial Sequence
  Synthetic Polypeptide
  MISC_FEATURE: VL-IgG Kappa
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 451 aa, Artificial Sequence
  Synthetic Polypeptide
  MISC_FEATURE: VH- IgG1
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 7 aa, Artificial Sequence
  Synthetic Polypeptide
  MISC_FEATURE: LC CDR1
FVGSSIH

SEQ ID NO: 26 - protein, 7 aa, Artificial Sequence
  Synthetic Polypeptide
  MISC_FEATURE: LC CDR2
KYASESM

SEQ ID NO: 27 - protein, 5 aa, Artificial Sequence
  Synthetic Polypeptide
  MISC_FEATURE: LC CDR3
QSHSW

SEQ ID NO: 28 - protein, 6 aa, Artificial Sequence
  Synthetic Polypeptide
  MISC_FEATURE: HC CDR1
IFSNHW

SEQ ID NO: 29 - protein, 10 aa, Artificial Sequence
  Synthetic Polypeptide
  MISC_FEATURE: HC CDR2
RSKSINSATH

SEQ ID NO: 30 - protein, 7 aa, Artificial Sequence
  Synthetic Polypeptide
  MISC_FEATURE: HC CDR3
NYYGSTY

SEQ ID NO: 31 - protein, 107 aa, Artificial Sequence
  Synthetic Polypeptide
  MISC_FEATURE: VL
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 32 - protein, 119 aa, Artificial Sequence
  Synthetic Polypeptide
  MISC_FEATURE: VH
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 33 - protein, 214 aa, Artificial Sequence
  Synthetic Polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 449 aa, Artificial Sequence
  Synthetic Polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK